(19) United States
(12) Patent Application Publication
(10) Pub. No.: US 2026/0345824 A1
(43) Pub. Date: Nov. 5, 2026

(54) SYSTEMS AND METHODS FOR AN INTRANASAL DRUG DELIVERY SYSTEM

(71) Applicants: Jim FALLON, Stuart, FL (US); John Lawrence MEE, Stuart, FL (US); Dean RADIN, Stuart, FL (US)

(72) Inventors: Jim FALLON, Stuart, FL (US); John Lawrence MEE, Stuart, FL (US); Dean RADIN, Stuart, FL (US)

(21) Appl. No.: 19/485,244

(22) PCT Filed: May 16, 2024
(86) PCT No.: PCT/US2024/029735

§ 371 Date: Nov. 17, 2025

Related U.S. Application Data
(60) Provisional application No. 63/502,618, filed on May 16, 2023.

(51) Int. Cl. A61K 48/00; A61K 9/00; A61K 41/00; A61P 25/22; C07K 14/705; C07K 14/72; C12N 9/22; C12N 15/11; C12N 15/86

(52) U.S. Cl. CPC A61K 48/005; A61K 9/0043; A61K 41/0047; A61K 48/0075; A61K 48/0083; A61P 25/22; C07K 14/70571; C07K 14/723; C12N 9/226; C12N 15/111; C12N 15/86; C12N 2310/20; C12N 2750/14143; C12N 2800/40

(57) ABSTRACT

Various aspects of this disclosure relate to methods of treating patients that present with mental health conditions by administering AAV9 vectors that edit genomic DNA in neurons to knockout a gene. The knockout may be mediated by a nuclease, such as Cas9, and a guide RNA. The AAV9 vectors may be administered intranasally.

### CROSS-REFERENCE TO RELATED APPLICATIONS

This application claims the benefit of U.S. Provisional Patent Application No. 63/502,618, filed May 16, 2023, the disclosure of which is incorporated, in its entirety, by this reference.

### SEQUENCE LISTING

This application includes a sequence listing named SEQUENCE_LISTING_109439.0031_2024-05-16.xml, which was created on May 16, 2024, which has a size of 4,000 bytes, and which is incorporated by reference in its entirety.

### TECHNICAL FIELD

The present disclosure relates generally to systems and methods for improving mental health and/or performance using an intranasally-administered neuron editing drug.

### BACKGROUND

The nasal pathway may represent a non-invasive administration route for active biopharmaceutical ingredients for local, systemic and central nervous system (CNS) action. Although the nasal epithelium appears to function as a tight barrier, the tightness of the intercellular junctional complex of the nasal mucosa may often be low due to leaky epithelial tissue. In addition, the extensive vascularization of the mucosa and lamina propria provide an optimal absorption surface for delivery of molecular therapies. The direct absorption of molecules through the trigeminal and olfactory pathways from the nasal cavity provides a direct entrance to the brain and may result in beneficial pharmacokinetic and pharmacodynamics profiles for CNS acting biologics. This route of administration is a promising new pathway for dosing highly potent and efficacious CNS-targeted biologics to reach the brain parenchyma by bypassing the blood-brain barrier. However, untargeted manipulation of CNS acting biologics via RNA or DNA affects the whole brain, potentially causing unwanted side effects and/or canceling out the desired effects. For example, although conventional forms of CRISPR can distinguish neurons from other types of cells in the body, they cannot differentiate between various areas in the brain.

As a result, improved methods of delivering CNS acting biologics which target specific areas of the brain are needed.

### SUMMARY

One aspect of the present disclosure includes methods of treating a patient with a mental health condition. These methods include administering a dose of a neuron-editing biologic intranasally to the patient. Such methods may also include administering a brain region activator to activate a target brain region for treatment. These methods further include transporting the neuron-editing biologic to active neurons within the target brain region using hemodynamics. The neuron-editing biologics may then normalize brain activity within the target brain region.

Another aspect of the present disclosure relates to methods of improving a patient's cognitive capacity. Such methods include administering a dose of a neuron-editing biologic intranasally to the patient. These methods also include administering a brain region activator to activate a target brain region. The methods further include transporting the neuron-editing biologic to the active neurons within the target brain region using hemodynamics. The neuron-editing biologics may then improve and/or enhance brain activity within the target brain region.

The foregoing has outlined rather broadly several features and technical advantages of examples of this disclosure so that the detailed description that follows may be better understood. Additional features and advantages will be described hereinafter. The concepts and specific examples disclosed herein may be readily utilized as a basis for modifying or designing other methods, constructs and/or systems for carrying out the same or similar purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a diagram listing a variety of neuron editing biologics in accordance with the principles of the present disclosure.

FIG. 2 is a diagram listing a variety of brain region activators in accordance with the principles of the present disclosure.

FIGS. 3A, 3B, 4A, and 4B illustrate an exemplary method of delivering a neuron-editing biologic drug intranasally in accordance with the present disclosure.

FIG. 5 is a map of plasmid pAAV9-Mecp2-spCas9, which is used to express a nucleic acid that encodes spCas9 with inverted terminal repeats (ITRs) for packaging the nucleic acid into an adeno-associated virus (AAV) vector.

FIG. 6A depicts green-fluorescence protein (GFP) reporter associated fluorescence on microelectrode array (MEA) plates, which indicates transfection of primary cortical neurons by an AAV serotype 9 (AAV9) vector carrying nucleic acid that encodes both guide RNA (gRNA) and GFP.

FIGS. 6B & 6C depict a decrease in number of spikes, number of bursts, and synchrony for primary cortical neurons treated with AAV9 vectors to knockout 5-hydroxytryptamine receptor 2A (5HT-2A receptor); differences between cells infected at a multiplicity of infection (MOI) of 200,000 (FIG. 6B at top-left panel; FIG. 6C at top panel) displayed statistical significance in each parameter relative to vehicle control (FIG. 6B at bottom-right panel; FIG. 6C at bottom panel).

FIG. 7A is a histogram that depicts decreases in number of spikes for primary cortical neurons treated with AAV9 vectors to knockout 5HT-2A receptor relative to vehicle control; cells treated with 40 microliters AAV9 corresponding to an MOI of 200,000 displayed a statistically significant decrease in number of spikes relative to vehicle control.

FIG. 7B is a histogram that depicts differences in number of bursts for primary cortical neurons treated with AAV9 vectors to knockout 5HT-2A receptor relative to vehicle control; cells treated with 40 microliters AAV9 corresponding to an MOI of 200,000 displayed a statistically significant decrease in number of bursts relative to vehicle control.

FIG. 7C is a histogram that depicts differences in synchrony index for primary cortical neurons treated with AAV9 vectors to knockout 5HT-2A receptor relative to vehicle control; cells treated with 40 microliters AAV9 corresponding to an MOI of 200,000 displayed a statistically significant decrease in synchrony index relative to vehicle control.

FIG. 8 is a histogram that depicts a statistically significant 8.47-fold decrease in HTR2A gene expression (which encodes 5HT-2A receptor) in brains of mice treated with AAV9 vectors to knockout 5HT-2A receptor.

FIG. 9 is a fluorescence microscopy image that depicts the expression of GFP throughout the CNS including olfactory bulb (OB), cortex, and sub-cortical areas including the interpeduncular nucleus (IPN) for a mouse treated with AAV9 vectors to knockout 5HT-2A receptor.

FIG. 10 is a histogram that depicts the quantification of anti-5HT-2A receptor immunohistochemistry-associated fluorescence in brains of mice treated with AAV9 vectors to knockout 5HT-2A receptor and in brains of vehicle-control mice.

FIG. 11 contains fluorescence microscopy images of the cortex, the Purkinje cell layer of the cerebellum, and pontine gray nuclei for mice treated with AAV9 vectors to knockout 5HT-2A receptor and corresponding images for vehicle-control mice. The left panels show fluorescence associated with GFP, which demonstrates transfection with the AAV9 vector that also encodes gRNA. The right panels depict fluorescence associated with 5HT-2A receptor. The nuclear stain DAPI is depicted in gray whereas GFP and 5HT-2A associated fluorescence is depicted in white. Mice that received AAV9 vectors to knockout 5HT-2A receptor displayed increased fluorescence associated with GFP and decreased fluorescence associated with 5HT-2A receptor relative to control in each set of images.

FIG. 12A-12C are graphs for mice subject to a marble-burying test. FIGS. 12A and 12B show statistically significant decreases in number of marbles buried by mice that received AAV9 vectors to knockout 5HT-2A receptor relative to vehicle control mice at 5-weeks and 7-weeks post-AAV9 treatment. FIG. 12C demonstrates that the treated and control mice displayed no difference in body weight.

FIG. 13A-13D are graphs for mice subject to a light/dark behavioral test. FIGS. 13A, 13B, and 13C show that mice treated with AAV9 vectors to knockout 5HT-2A receptor displayed a statistically significant increase in time spent in the lit box, entries into the lit box, and locomotion in the lit box, respectively, relative to vehicle-only controls. FIG. 13C shows that mice treated with AAV9 vectors to knockout 5HT-2A receptor displayed a similar amount of time spent in the lit box relative to positive-control mice that received diazepam.

FIG. 13D depicts the number of entries into the lit box relative for AAV9-treated mice relative to positive-control mice that received diazepam, which indicates that the AAV9-treated mice exited the lit box less frequently than diazepam-treated mice.

FIG. 14 is a graph for mice subject to an elevated plus maze (EPM) test and depicts a statistically significant decrease in entries into the closed arm of the EPM for mice treated with AAV9 vectors to knockout 5HT-2A receptor relative to vehicle controls.

FIG. 15 is an image of an electrophoresis gel from a T7 endonuclease 1 (T7E1) assay on gRNA designed to knockout 5HT-2A receptors in humans as well as negative controls. The gRNA “sgRNA8,” which includes the targeting sequence SEQ ID NO: 3, displayed appropriate performance in the assay as evidenced by two shorter nucleic acids identified by arrows in FIG. 15, and this gRNA was selected for use in AAV9 vectors for human trials.

FIGS. 16A & 16B are images of human iPSC cell lines that contain 5HT-2A receptor knockouts generated using Cas9 and a gRNA that contains the targeting sequence SEQ ID NO:3.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

This description provides examples, and is not intended to limit the scope, applicability or configuration of the invention. Rather, the ensuing description will provide those skilled in the art with an enabling description for implementing embodiments of the invention. Various changes may be made in the function and arrangement of elements.

Thus, various embodiments may omit, substitute, or add various procedures or components as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following systems, methods, and devices may individually or collectively be components of a larger system, wherein other procedures may take precedence over or otherwise modify their application.

The detailed description of exemplary embodiments herein makes reference to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

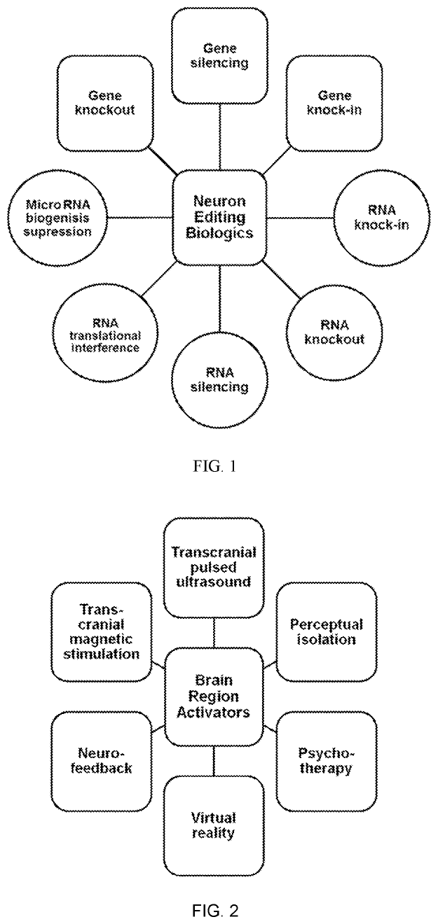

As used herein, neuron editing biologics are biopharmaceuticals, in some cases protein-based, or peptide-based biopharmaceuticals, which can modify a neuron's DNA or RNA. FIG. 1 illustrates eight examples of neuron-editing biologics which are discussed below. As listed, these may include gene knockout biologics, gene silencing biologics, and/or gene knock-in biologics.

Gene knockout biologics may include a catalytically-active gene-editing endonuclease(s) complexed with a synthetic guide RNA. Gene silencing biologics may include a catalytically-inactive gene-editing endonuclease(s) complexed with a synthetic guide RNA. Gene knock-in biologics may include a gene-editing endonuclease(s) complexed with a gene-expression inhibiting nucleotide and a synthetic guide RNA.

As used herein, transcriptional engineering biologics may include RNA knockout, RNA silencing, RNA knock-in, RNA translational interference, and Micro-RNA biogenesis suppression compounds/systems. RNA knockout compounds may include a catalytically-active RNA-editing ribonuclease complexed with a single guide RNA to alter RNA nucleotides to repress gene translation. RNA silencing compounds may include is a catalytically-inactive RNA-editing ribonuclease complexed with a single guide RNA to bind to RNA nucleotides to repress or enhance gene translation. RNA knock-in biologic may include a catalytically-inactive RNA-editing ribonuclease complexed with a single guide RNA and a deaminase enzyme to cause RNA nucleobase substitutions which result in translational interference. RNA translational interference compounds may include an RNA-editing ribonuclease complexed with an RNA-expression inhibiting nucleotide and a single guide RNA to alter RNA nucleotides to cause translational interference. Micro-RNA biogenesis suppression compounds may include a catalytically-active ribonuclease complexed with a single guide RNA to alter nucleotides in biogenesis processing sites for micro-RNA used in the translation of genes in order to reduce their expression.

As used herein, brain region activators include methods and techniques for focusing and concentrating the neuronal activity in a patient's brain into a specific, targeted area of the brain. FIG. 2 lists six examples of brain region activators which are explained below. Others may be known to one skilled in the art.

As used herein, transcranial pulsed ultrasound is a technique which uses low-power, low-frequency ultrasound to stimulate high neuron activity in the brain. It may be directed to any brain region and precisely focused to areas as small as several cubic millimeters.

As used herein, transcranial magnetic stimulation is a form of neurostimulation which uses a shallow magnetic field to induce electric current to flow in small targeted regions near the surface of the brain.

As used herein, perceptual isolation is the deliberate removal of stimuli from the senses of a subject/patient. Examples of perceptual isolation methods include sleep masks, white noise, soundproofing, and/or floatation tanks.

As used herein, neurofeedback is a type of biofeedback that measures brain waves to produce a signal that can be used as feedback to teach self-regulation of brain function. Patients can be trained to alter their brain activity to increase performance on certain tasks, which in turn changes the signal and increases cerebral blood flow to a specified region of the brain.

As used herein, virtual reality may be an immersive, interactive, computer-generated experience which occurs in a simulated environment including auditory and visual feedback.

As used herein, psychotherapy is a wide field encompassing the use of any one or more of hundreds of different methods and techniques to improve an individual's well-being, behavior and mental health, including cognitive therapy and other forms of therapies.

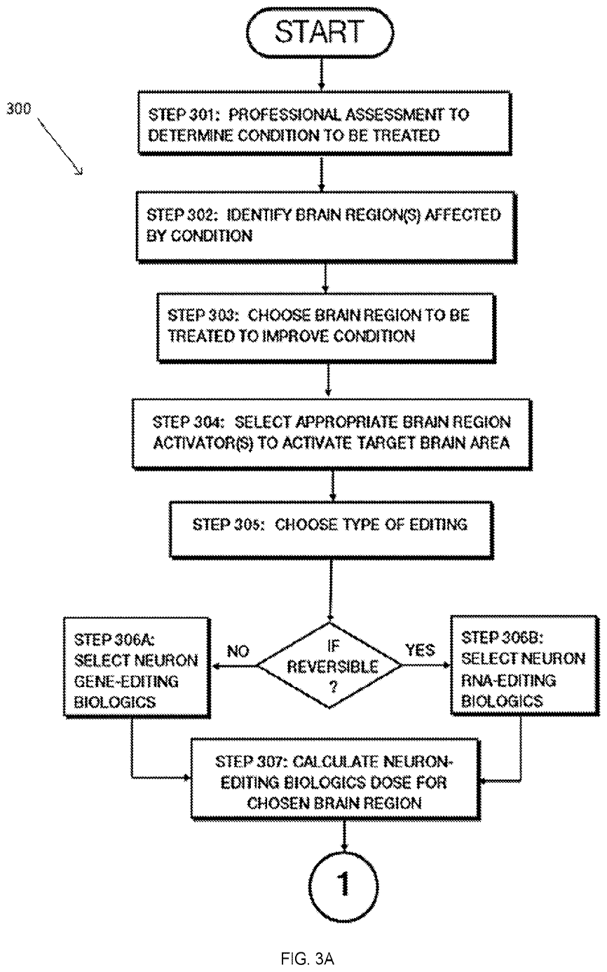

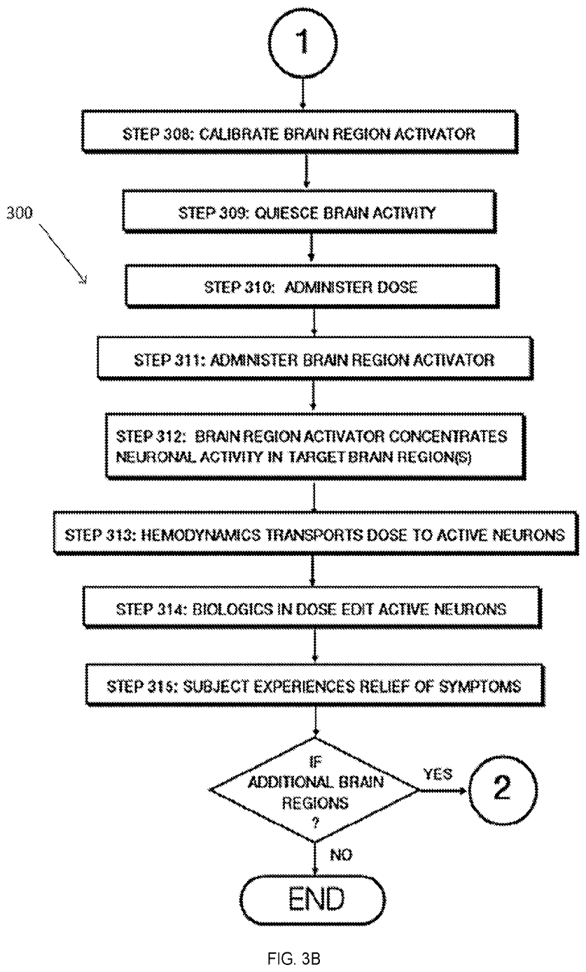

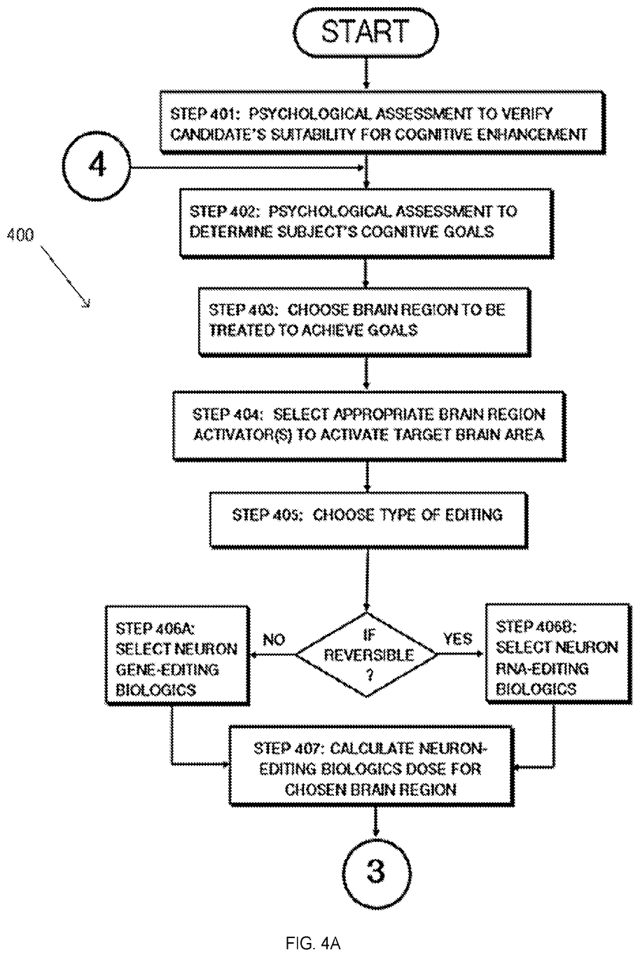

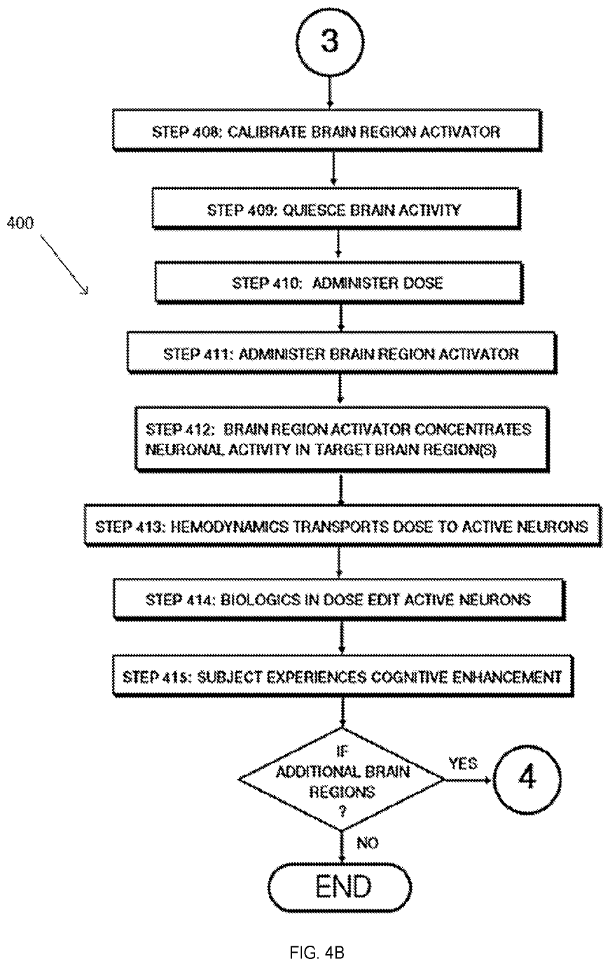

Referring next to FIGS. 3A, 3B, 4A, and 4B, a flow diagram of a method 100 of delivering a drug intranasally is provided. Specifically, the diagram describes a method 100 for intranasally delivering gene therapies which modify neuron electrodynamic excitability in the brain's limbic region to treat common mental health issues such as stress, anxiety, depression, PTSD and ADD, as well as personality disorders. More specifically, as described herein, the method 100 enables a region of the patient's brain to be activated such that a drug delivered intranasally can target the specific activated region of the brain to treat a specific mental health condition.

Intranasal delivery allows biologics to enter the CNS through the rostral migratory stream, which is a specialized migratory route along which neuronal precursors that originate in the subventricular zone of the brain migrate to reach the olfactory bulb. Intranasal delivery introduces biologics into the rostral migratory stream and also allows perineuronal and perivascular transit of the biologics throughout the brain. Regardless of the biological mechanism of transit, intranasal delivery allows the distribution of biologics throughout the entire brain, including the olfactory bulb, cortex, subcortical regions, cerebellum, and pons as shown, for example, in FIGS. 9 & 11. Intranasal delivery also allows the distribution of biologics, for example, in the thalamus, hypothalamus, hippocampus, and amygdala. It is understood that introduction of agents introduced in this manner travel not just transvascularly, but also transneuronally, from one neuron to another.

Common mental health issues such as stress, anxiety, depression and inattention are all experimentally correlated with overactive neurons in specific areas of the limbic system. Neuron activity levels in these areas can be normalized through gene therapies which alter a neuron's structure to lower its electrical excitability. Although conventional forms of CRISPR can distinguish neurons from other types of cells in the body, they cannot differentiate between various areas in the brain. The illustrated method 100 allows for delivery of neuron-editing biologics to targeted regions and connectomes of the brain to achieve specific neurological treatment or cognitive enhancement goals.

Directing CRISPR molecules to specific types of cells is known as vectoring. Current CRISPR vectoring technology can target neurons, but it cannot provide navigational specificity to individual areas within the brain. It is demonstrated herein, however, that biologics such as CRISPR biologics may be directed in a patient's bloodstream along with glucose and oxygen to specific active areas in the brain by the principles of hemodynamics.

Glucose and oxygen are the fuel which powers neuronal activity. The more active a neuron is, the faster it burns glucose and oxygen. Unlike other types of cells, neurons cannot store glucose, so their supply must be immediately replenished as soon as it is consumed. A circulatory system principle known as hemodynamics ensures the vital nutrients of glucose and oxygen are rapidly delivered to active neurons.

Hemodynamics principles can be used to direct CRISPR into active neurons which are firing. By employing a selective brain region activation process to concentrate the patient's neural activity into a targeted brain region, passive hemodynamics techniques can be used to draw cerebral blood flow containing CRISPR neuron editing biologics into the region. By employing a selective brain region activation process to concentrate the patient's neural activity into a targeted brain region, the method 100 can use passive hemodynamics to draw cerebral blood flow containing CRISPR neuron editing biologics into the region. Additionally, ligand vectors may be used which include targeting peptide nanoparticles which can bind to receptors that are unique to different cell types and thus to deliver gene or transcriptional editing cargo to them.

A plurality of brain region activation processes can be used by cognitive neuroscience professionals to serve different applications and objectives. Method 100 outlines a framework and platform to enable cognitive neuroscience professionals to deploy a variety of applications that target specific regions of the brain to treat specific mental health conditions.

Generally, the method 100 generally includes the steps of (1) assessment of the patient/subject to determine issues and establish goals; (2) identification of brain regions to be treated; (3) pre-treatment/cognitive therapy of the patient; (4) prescription and administration of selected biologics to the patient; and (5) post-treatment/cognitive therapy.

In specific embodiments, the method 100 enables intranasal delivery of neuron-editing biologics to the olfactory epithelium via a swab or nasal inhaler. The olfactory bulb pathways transport the biologics directly to the limbic lobe and the neuron-editing biologics target the limbic system to treat mental health conditions such as, but not limited to, inattention, stress, anxiety and depression. In some instances, the method 100 may be used to attenuate hyperactive or hypoactive neurons in the limbic system which are responsible for inattention (ventral posterior cingulate cortex (PCC)), stress (amygdala, hippocampus), anxiety (amygdala), and depression (amygdala, hippocampus).

Advantages of method 100 include (1) limiting biologics delivery to the region of interest to minimize dosing requirements and avoid affecting other areas of the brain; (2) avoid damage to biologic agents by avoiding transit which would expose them to digestive enzymes or immune system response by delivering them intranasally; (3) alleviating hippocampal and amygdaloid hyperactivity or hypoactivity which may also improve memory and could potentially halt the progression of dementia and extend longevity; and (4) in addition to one-time DNA edits, some applications may involve multiple iterations of DNA edits. For example, the multiple iterations of DNA edits may include layered DNA edits, where the edit is divided into multiple smaller doses, or temporary RNA edits, which are repeated over time. AAV vectors have been used for editing neurons, but after one application, the body develops an immune response to the AAV vectors, preventing further uses. However, using viral vectors for multiple iterations is feasible with intranasal delivery because it transports gene editing payloads directly into the brain without activating an immune system response.

Once inside the limbic lobe, the method 100 includes three further methods to direct neuron-editing biologics to a target region of the patient's brain. First, actively-directed hemodynamic vectoring can transport the biologics to specific areas of the patient's brain which are energized by brain region activators. Second, passive hemodynamic vectoring will naturally transport the biologics to the most active neurons of the patient's brain. Finally, ligand vectors including targeting peptide nanoparticles designed to direct editing biologics to neuron cell types may be used to direct them to specific brain regions of the patient's brain. Hemodynamic and ligand vectoring are complementary therapies. Ligand vectors may be pre-programmed to statically treat specific topographical areas of brainwave activity imbalance identified by a neuroimaging assessment. Hemodynamic vectors can dynamically treat in real-time systemic electrophysiological activity imbalances stimulated, for example, by psychotherapy, virtual reality, and/or perceptual events, as well as statically targeting specific regions of the brain. However, ligand vectors can transport biologics only to neurons which have the receptor types of interest by sensing proteins which are unique to each receptor type, limiting the types of neurons that can be treated with ligand vectors. Method 100 enables biologics to edit any type of neuron by activating the neuron prior to intranasal delivery of the biologic, enabling the biologic to edit the types of neurons that ligand vectoring cannot edit.

Thus, method 100 may include a specific-purpose neurological condition reliever which works on individual conditions (e.g., inattention, stress, anxiety, depression) by genetically altering neuron structure in order to modify neuron electrodynamics. Method 100 relieves the conditions by intranasal delivery of neuron-editing biologics to the limbic system in combination with (1) actively-directed hemodynamic vectoring, (2) passive hemodynamic vectoring, and/or (3) ligand vectors.

Method 100 may include either or both of treatment 300 and enhancement 400 steps, as described in co-pending U.S. patent application Ser. No. 16/272,138, which is incorporated herein by reference. Treatment 300 generally includes treating a specific mental health condition and enhancement 400 generally includes general-purpose cognitive enhancements. Treatment 300 and enhancement 400 may be used together or separately as determined by a medical professional. Using the method 100, treatment 300, and enhancement 400 may be used to treat a variety of neurological medical conditions. Specifically, the method 100, including treatment 300, and enhancement 400 steps may be used to treat a wide variety of mental health conditions. In some specific instances, the method 100, treatment 300, and/or enhancement 400 steps may be used to treat Attention Deficit Disorder (ADD) and Attention Deficit Hyperactivity Disorder (ADHD), Post Traumatic Stress Disorder (PTSD), Obsessive Compulsive Disorder (OCD), stress disorders, anxiety, depression, sleep conditions, memory conditions, personality disorders, schizophrenia, concussions and/or head injuries, Tourette Syndrome, psychosomatic issues, tinnitus, conscious awareness issues, inattention, mind wandering, cravings, mental focus issues, concentration issues, mindfulness issues, meditation issues, and intuition issues.

Treatment steps 300 may include treatment steps 301-317 that enable a medical professional to treat a mental illness such as ADD/ADHD, PTSD, OCD, stress disorders, anxiety, depression, sleep conditions, memory conditions, concussions and/or head injuries, Tourette Syndrome, psychosomatic issues, and/or tinnitus. However, before treatment 300 can begin, a medical professional prepares the patient with pre-treatment therapy. During pre-treatment therapy, the medical professional may set expectations which foster positive beliefs about change by employing psychological counseling techniques such as cognitive behavioral therapy, mindfulness-based stress reduction, neuro-linguistic programming, meditation, and/or visualization.

Treatment phase 300 may include step 301: a professional assessment to determine a condition to be treated. During step 301, a medical professional may conduct an assessment of the patient to identify a specific neurological issue, condition, and/or disorder to be treated. The medical professional can be a human or in some instances may be an AI computer program.

Treatment phase 300 may also include step 302: identification of brain regions affected by the identified neurological issue, condition, and/or disorder to be treated. Neurological conditions and disorders may cause under- or over-activation in multiple areas in the brain. The medical professional may order a neuroimaging study, such as, without limitation, an fMRI, PET or SPECT brain scan, to pinpoint area(s) in the patient's brain which may be over- or under-activated. The brain scan may show abnormal activity in one or more regions, depending on the condition to be treated. Brain regions typically affected by 12 common neurological conditions are shown in Table 1 below.

Treatment phase 300 may further include step 303: selecting a brain region to be treated to improve the condition. The medical professional may establish treatment priorities according to the degree of over- or under-activation of each region as revealed in the neuroimaging study, in some instances beginning with the most over- or under-activated area.

Treatment phase 300 may further include step 304: selecting an appropriate brain region activator to activate the target brain regions. The brain region activator activates the selected brain region to be treated to enable the gene therapies to target the selected brain region. Specifically, the brain region activator is selected to activate a specific region of the brain such that the gene therapies are absorbed into the selected brain region. Table 1 illustrates 6 examples of brain region activators which may be suitable for any one or more of at least 12 neurological treatment applications. Other brain region activation methods can be devised and employed by those skilled in the art. The activator examples shown are: transcranial pulsed ultrasound (TPU), transcranial magnetic stimulation (TMS), neurofeedback, perceptual isolation, virtual reality (VR), and psychotherapy.

As briefly discussed above, TPU may involve the use of low intensity, low frequency ultrasound (LILFU) to stimulate the patient's brain and may be effective in a variety of applications because TPU can be focused in almost any area of the brain with a precision down to a few square millimeters. Furthermore, TPU may in some instances also increase the permeability of the brain's blood brain barrier, allowing for more effective transport of neuron-editing biologics in the bloodstream to the target brain regions. TPU may also be used in combination with other tools and techniques such as, but not limited to, heat-sensitive CRISPR vectors, such as lipid nanoparticles, to provide very precise delivery targeting.

As also briefly discussed above, TMS may include a noninvasive form of brain stimulation in which a changing magnetic field is used to cause electric current at a specific area of the brain through electromagnetic induction and has been used successfully in the treatment of depression. However, TMS' effective range is maximal to neurons in the cortex, near the brain's surface with progressively less effect deep to the cortex. Neurofeedback is a technique involving the use of a type of biofeedback that presents real-time feedback from brain activity to a patient/subject in order to reinforce healthy brain function through operant conditioning and has been shown to be effective across a wide spectrum of applications. Given use of the appropriate techniques, neurofeedback can be used to selectively activate almost any area in the brain of a subject.

As defined above, perceptual isolation includes the deliberate reduction or removal of stimuli from one or more of the senses of a subject and may be used in applications addressing the default mode network (DMN) (posterior cingulate cortex, medial prefrontal cortex, angular gyrus) to quiesce neural activity in other areas of the brain and concentrate neural activity into the DMN.

VR may include simulating in whole or in part, an experience that can be similar to or completely different from the real world. In some instances, VR has been used in applications treating aversive memories. A VR simulation can be used to strongly reactivate the memory and using methods of the invention, genetic or transcriptional therapies can be used to attenuate the neuronal activity associated with the reactivated, unwanted memories.

Psychotherapy may include the use of psychological methods, particularly when based on regular personal interaction, to help a person change behavior, increase happiness, and/or overcome problems and can have efficacy in applications treating aversive memories, stress, anxiety, and/or depression. As discussed above, by reactivating unwanted memories or conditions, psychotherapy can be used with the methods of this disclosure to induce genetic or transcriptional therapies to attenuate their associated neuronal activity.

Treatment phase 300 may also include step 305: selecting a type of editing. According to methods provided herein, a medical professional may select a type of editing for treatment of the identified condition. For example, the medical professional may select one of at least transcriptional editing and genetic editing. Transcriptional editing may be used to produce a reversible, temporary result for testing purposes. Genetic editing is selected when the patient is ready for a permanent treatment.

Treatment phase 300 may further includes step 306: selecting a neuron-editing biologic. In some instances, the brain region selected for treatment may be over- or under-active. If the region is underactive, the medical professional may select neuron-editing biologics which raise neuron excitability to increase activity in the region. If the region is overactive, the medical professional may select neuron-editing biologics which reduce neuron excitability in order to lower neuronal circuitry in the region. In some methods, a neuron's excitability can be reduced by decreasing its receptor population. One method for decreasing the receptor population is to edit gene HTR2A or its transcriptional or translational pathways to reduce HTR2A's expression, thus lowering the population of serotonin 2A receptors, as described in co-pending U.S. patent application Ser. No. 15/970,037, which is incorporated herein by reference. If ligand vectoring is to be used, the medical professional selects neuron-editing biologics with ligand vectors targeting the brain area to be edited.

Treatment phase 300 may also include step 307: calculating a dosage of neuron-editing biologics for the selected brain region. In this step, the medical professional may calculate an appropriate dose based on a plurality of factors including, but not limited to, the age, weight, gender, and/or physical condition of the patient, the condition to be treated, and/or the region of the patient's brain to be targeted. An example of a dosing formula is included in co-pending U.S. patent application Ser. No. 15/970,037, which is incorporated herein by reference. If ligand vectoring is being used in the method, steps 308 and 309 of the treatment phase may be omitted, and a practitioner may instead proceed to step 310 described below.

Treatment phase 300 may further include step 308: calibrating the brain region activator. The medical professional calibrates the brain region activator method chosen in Step 304 discussed above to address the individual patient's brain region targeted for treatment. For transcranial pulsed ultrasound or transcranial magnetic stimulation, for example, the medical professional focuses the device on the target area in the patient's brain. For neurofeedback, the medical professional selects a neurofeedback program designed to address the patient's target brain region specifically. For perceptual isolation, the medical professional may select the stimuli reduction method to be used. For virtual reality, the medical professional may choose a VR simulation designed to evoke a particular memory or emotion related to the patient's issue. For psychotherapy, the medical professional may select a session protocol designed to evoke the patient's unwanted memory or condition.

The treatment phase 300 may also include step 309: quiesce brain activity. In this step, the patient may be placed in a resting state free from distractions to minimize brain activity to minimize activity in and/or deactivate all brain regions except the default mode network.

Treatment phase 300 may further include step 310: administering the calculated dose of the neuron-editing biologics to the subject. In this step, the neuron-editing biologics may be administered intranasally to the patient via swab or nasal inhaler.

If ligand vectors are being used, steps 311-313 may be omitted and a practitioner may instead proceed to step 314 described below.

Treatment phase 300 may also include step 311: administering the selected brain region activator to activate the target brain region(s). In this step, the medical professional administers the brain region activator method selected for the patient in Step 304 discussed in detail above.

Treatment phase 300 may further include step 312: concentrating neural activity in the target brain region using the brain region activator. The brain region activator is used by the practitioner to concentrate neuronal activity in the target brain region(s) of the patient/subject receiving treatment.

Treatment phase 300 may also include step 313: transporting the neuron-editing biologics to the active neurons within the target brain region using hemodynamics. As discussed above, cerebral blood flow containing glucose, oxygen, and neuron editing biologics is transported to active neurons in the targeted area of the patient's brain by hemodynamics principles.

Treatment phase 300 may further include step 314: editing the active neurons using the neuron-editing biologics. Neuron-editing biologics may here be used to transfect neurons in the target area of the patient's brain and edit the neurons' DNA or RNA to normalize activity in the region as determined by the medical professional in the previous steps.

Treatment phase 300 may also include step 315: reducing symptoms of the condition. Normalized brain region activity relieves the symptoms of the patient's neurological condition.

Treatment phase 300 may further include step 316: iterating steps 302-315 as needed. If multiple brain areas are indicated for treatment, the medical professional may return to step 302 discussed above to address the next area of priority. The brain scan performed at this juncture may be used to verify/assess the potentially normalized brain activity in the region just treated.

Treatment phase 300 may also include step 317: conducting post-treatment therapy. Post-treatment therapy may be used to instill appropriate mind and life practices in the subject to reinforce the gene therapy's effects by fostering supportive behavioral and epigenetic factors. Post-treatment therapy can employ psychological counseling protocols such as cognitive behavioral therapy, mindfulness-based stress reduction, neuro-linguistic programming, meditation, visualization, and/or other techniques.

Table 1 illustrates the practical application of the treatment 300 in treating twelve examples of common neurological conditions:

TABLE 1
practical applications of the treatment phase 300 of the method
100 in treating twelve examples of common neurological conditions.
Treatment

| | | Brain region activator | | | | | |
|---|---|---|---|---|---|---|---|
| | | Transcranial | Transcranial | | Perceptual | Virtual | |
| Issue | Main Brain regions affected | ultrasound | magnetic stim. | Neurofeedback | isolation | reality | Psychotherapy |
| 1. ADD/ADHD | Amygdala, ventromedial prefrontal | ● | | ● | | | |
| | cortex, hippocampus (under- | | | | | | |
| | activated) | | | | | | |
| | Dorsal attention and default mode | ● | | ● | ● | | |
| | networks (over-activated) | | | | | | |
| 2. Traumatic | Amygdala, hippocampus | ● | | ● | | ● | ● |
| memory/PTSD | Ventromedial prefrontal cortex | ● | | ● | | ● | ● |
| 3. OCD | Orbital gyrus, caudate nucleus, | ● | | ● | | | |
| | dorsal anterior cingulate cortex | | | | | | |
| 4. Stress disorders | Amygdala, hypothalamus | ● | | ● | | | ● |
| 5. Anxiety | Amygdala | ● | | ● | | | ● |
| 6. Depression | Posterior cingulate cortex, | ● | ● | ● | ● | | |
| | prefrontal cortex (underactive) | | | | | | |
| | Amygdala, hippocampus, anterior | ● | | ● | | | |
| | cingulate cortex (overactive) | | | | | | |
| 7. Sleep issues | Prefrontal and parietal cortex, | ● | | ● | ● | | |
| | precuneus, anterior cingulate, | | | | | | |
| | mesial temporal, thalamus and | | | | | | |
| | hypothalamic arousal centers, | | | | | | |
| | default-mode network | | | | | | |
| 8. Memory | Hippocampus, amygdala | ● | | ● | | | |
| concerns | | | | | | | |
| 9. Concussions | Frontal and temporal lobes | ● | | ● | | | |
| 10. Tourette | Basal ganglia | ● | | | | | |
| Syndrome | | | | | | | |
| 11.Psychosomatic | Amygdala, hippocampus | ● | | ● | | | ● |
| issues | Ventromedial prefrontal cortex | ● | | ● | | | ● |
| 12. Tinnitus | Auditory cortex | ● | | | | | |

In applying the methods of this disclosure, a medical practitioner should be aware of the characteristics of the neurological and/or psychiatric. condition being treated, and devise specific treatment phase 300 methodologies as discussed with respect to the twelve examples provided in Table 1 below:

First, Attention Deficit Disorder (ADD) and Attention Deficit Hyperactivity Disorder (ADHD) may involve altered activation of the amygdala, ventromedial prefrontal cortex and/or hippocampus, depending on the ADD or ADHD subtype. Activity in these areas can be corrected by administering neuron-editing biologics designed to alter neuron excitability, in conjunction with transcranial pulsed ultrasound or neurofeedback brain region activation techniques which direct the biologics to the amygdala, ventromedial prefrontal cortex and/or hippocampus via hemodynamic vectoring.

ADD and ADHD may also involve overactivation of the dorsal attention and default mode networks. Activity in these areas can be reduced by administering neuron-editing biologics designed to alter-neuron excitability in conjunction with transcranial pulsed ultrasound, neurofeedback and/or perceptual isolation brain region activation techniques which direct the biologics to the dorsal attention and/or default mode networks via hemodynamic vectoring.

In some subjects, Post-Traumatic Stress Disorder (PTSD) may involve overactivation of the amygdala and/or hippocampus. These areas can be corrected by administering neuron-editing biologics for altering neuron excitability in conjunction with transcranial pulsed ultrasound, neurofeedback, virtual reality or psychotherapy brain region activation techniques selected to direct the biologics to the amygdala and/or hippocampus via passive hemodynamic vectoring.

PTSD may also be characterized by altered activation of the ventromedial prefrontal cortex. Activity in this area can be corrected by neuron-editing biologics for changing neuron excitability in conjunction with transcranial pulsed ultrasound, neurofeedback, virtual reality and/or psychotherapy brain region activation techniques which direct the biologics to the ventromedial prefrontal cortex via hemodynamic vectoring.

Neuroimaging studies of people with Obsessive Compulsive Disorder (OCD) have revealed hyperactivity in the dorsal anterior cingulate cortex, orbital gyrus and caudate nucleus. Activity in these areas can be normalized by administering neuron-editing biologics for reducing neuron excitability, together with transcranial pulsed ultrasound or neurofeedback brain region activation techniques which direct the biologics to the dorsal anterior cingulate cortex, orbital gyrus and/or caudate nucleus via hemodynamic vectoring.

Individuals with stress disorders exhibit hyperactivity in the amygdala and hypothalamus. These areas can be brought into balance by administering neuron-editing biologics for altering neuron excitability, combined with transcranial pulsed ultrasound, neurofeedback or psychotherapy brain region activation techniques which direct the biologics to the amygdala and/or hypothalamus via hemodynamic vectoring.

Individuals experiencing anxiety exhibit hyperactivity in the amygdala, which can be normalized by administering neuron-editing biologics for altering neuron excitability, combined with transcranial pulsed ultrasound, neurofeedback or psychotherapy brain region activation techniques which direct the biologics to the amygdala via hemodynamic vectoring.

Depression involves disrupted activation of the posterior cingulate cortex and prefrontal cortex. Activity in these areas can be corrected by administering neuron-editing biologics designed to normalize neuron excitability, in conjunction with transcranial pulsed ultrasound, transcranial magnetic stimulation, neurofeedback, and/or perceptual isolation brain region activation techniques which direct the biologics to the posterior cingulate cortex and/or prefrontal cortex via hemodynamic vectoring.

Depression is also associated with altered activation of the amygdala, hippocampus and anterior cingulate cortex. Activity in these areas can be normalized by administering neuron-editing biologics designed to changing neuron excitability, in conjunction with transcranial pulsed ultrasound or neurofeedback brain region activation techniques which direct the biologics to the amygdala, hippocampus, and/or anterior cingulate cortex via hemodynamic vectoring.

Insomnia and other sleep issues can involve altered activation of the prefrontal and parietal cortex, precuneus, anterior cingulate, mesial temporal, thalamus and hypothalamic arousal centers, and the default-mode network. Activity in these areas can be normalized by administering neuron-editing biologics for correcting neuron excitability, in conjunction with transcranial pulsed ultrasound, neurofeedback and/or perceptual isolation brain region activation techniques which direct the biologics to the prefrontal and parietal cortex, precuneus, anterior cingulate, mesial temporal, thalamus and hypothalamic arousal centers, and/or the default-mode network via hemodynamic vectoring.

Neuroimaging studies of individuals with memory problems reveal subnormal activity in the hippocampus and amygdala. These areas can be normalized by administering neuron-editing biologics for altering neuron excitability, in conjunction with transcranial pulsed ultrasound or neurofeedback brain region activation techniques which direct the biologics to the hippocampus and/or amygdala via hemodynamic vectoring.

Head injuries may pathologically change neuronal activity in the frontal and temporal lobes. Corrected activity in these areas can be promoted by administering neuron-editing biologics for increasing neuron excitability, together with transcranial pulsed ultrasound or neurofeedback brain region activation techniques which direct the biologics to the frontal and/or temporal lobes via hemodynamic vectoring.

Studies of individuals with Tourette Syndrome show abnormal activity in the basal ganglia. Activity can be normalized by administering neuron-editing biologics for increasing neuron excitability, along with transcranial pulsed ultrasound brain region activation techniques which direct the biologics to the basal ganglia via hemodynamic vectoring.

Psychosomatic issues may involve overactivation of the amygdala and hippocampus. These areas can be normalized by administering neuron-editing biologics for changing neuron excitability, in conjunction with transcranial pulsed ultrasound, neurofeedback, or psychotherapy brain region activation techniques which direct the biologics to the amygdala and/or hippocampus via hemodynamic vectoring.

Psychosomatic issues may also cause altered activation of the ventromedial prefrontal cortex. Activity in this area can be corrected by neuron-editing biologics for normalizing neuron excitability, in conjunction with transcranial pulsed ultrasound, neurofeedback, or psychotherapy brain region activation techniques which direct the biologics to the ventromedial prefrontal cortex via hemodynamic vectoring.

Tinnitus/persistent ringing in the ears is caused by an overactivated auditory connections. Activity can be reduced by administering neuron-editing biologics for decreasing neuron excitability, along with transcranial pulsed ultrasound brain region activation techniques which direct the biologics to the auditory cortex via hemodynamic vectoring.

The enhancement phase 400 of the methods 100 disclosed herein may include enhancement steps 401-417 that enable a medical professional to enhance a subject's cognition, including areas such as, without limitation, conscious awareness issues, inattention, mind wandering, cravings, mental focus issues, concentration issues, mindfulness issues, meditation issues, and/or intuition issues. As a typical initial step before the enhancement phase 400 is initiated, a medical professional prepares the patient with pre-treatment therapy. During this pre-treatment therapy, the medical professional may set expectations which foster positive beliefs about change by employing psychological counseling techniques such as cognitive behavioral therapy, mindfulness-based stress reduction, neuro-linguistic programming, meditation, and/or visualization.

Enhancement phase 400 may include step 401: professional psychological assessment to verify a patient's suitability for cognitive enhancement. General-purpose genetic cognitive enhancement is generally suitable for adults in sound mental and emotional health. As a result, during step 401, a medical professional may conduct a psychological assessment of the patient to screen out candidates who do not meet this criteria, such as, for example, individuals with alcohol or substance abuse, bipolar disorder, depression, schizophrenia or other psychological conditions or disorders. The assessment also ensures the candidate is not currently taking any drugs, medications or substances that could interfere with the normal, natural functioning of their brain; such as, for example, certain prescription drugs, alcohol, caffeine, nicotine, cannabis, nootropics, ginseng or other similar substances or herbal preparations. In some instances, the medical professional who conducts this step can be a human or an AI computer program.

Enhancement phase 400 may also include step 402: conduct a psychological assessment to determine the patient's cognitive goals. The medical professional may conduct a psychological assessment to ascertain the patient's cognitive enhancement goals. This assessment may cover several topics, including the type of cognitive enhancement the patient desires and whether the cognitive upgrade is to be permanent or temporary.

Enhancement phase 400 may further include step 403: selecting a brain region to be treated to achieve the cognitive goals previously identified. Target brain regions for thirteen specific cognitive enhancement applications are illustrated in Table 2. Genetic or transcriptional engineering can be used to optimize neuron performance in these regions in order to achieve the patient's goals. Depending on the application, optimization may involve increasing or decreasing neuronal activity. If ligand vectors are being used, step 404 may be omitted, and a medical professional may instead proceed to step 405 described below.

Enhancement phase 400 may further include step 404: selecting an appropriate brain region activator to activate the target brain regions. The brain region activator activates the selected brain region to be treated to enable the gene therapies to target the selected brain region. Specifically, the brain region activator is specifically selected to activate a specific region of the brain such that the gene therapies are directed to and absorbed into the selected brain region. Table 2 illustrates six examples of brain region activators suitable for thirteen exemplary cognitive enhancement applications. Other brain region activation methods can be devised by those skilled in the art based on the teachings provided herein. The activator examples shown are identical to the ones described in Step 304 described above.

Enhancement phase 400 may also include step 405: selecting a type of editing. The medical professional may select a type of editing for treatment of the identified condition. For example, the medical professional may select one of at least transcriptional editing and genetic editing. Transcriptional editing may be used to produce a reversible, temporary result in a patient, such as for testing purposes, allowing the patient and/or practitioner to evaluate the effects of the enhancement on a temporary basis. Genetic editing may be selected when the patient is ready for a permanent treatment.

Enhancement phase 400 may further include step 406: selecting a neuron-editing biologic. The brain region selected for enhancement may be over- or under-active. If the region is underactive, the medical professional may select neuron-editing biologics which raise or lower neuron excitability to normalize activity in the region. If the region is overactive, the medical professional may select neuron-editing biologics which reduce neuron excitability in order to normalize the region. For example, a neuron's excitability can be reduced by decreasing its receptor population, which raises its electrical resistance. One way decreasing the receptor population is to edit gene HTR2A or its transcriptional or translational pathways to reduce HTR24's expression, which will lower the population of serotonin 2A receptors, as described in co-pending U.S. patent application Ser. No. 15/970,037, which is incorporated herein by reference. If ligand vectoring is to be used, the medical professional may select neuron-editing biologics with ligand vectors targeting the brain area to be edited.

Enhancement phase 400 may also include step 407: calculating a neuron-editing biologics dose for the selected brain region. The medical professional calculates an appropriate does for the neuron-editing biologics based on a plurality of factors including, but not limited to, the age, weight, gender, and/or physical condition of the patient, the condition to be treated, and/or the region of the patient's brain to be targeted. An example of a dosing formula is included in co-pending U.S. patent application Ser. No. 15/970,037, which is incorporated herein by reference. If ligand vectoring is being used, skip steps 408 and 409 and proceed to step 410 described below.

Enhancement phase 400 may further include step 408: calibrating the brain region system specific connectome. The medical professional calibrates the brain region method selected in Step 404 to address the patient's targeted brain region. For transcranial pulsed ultrasound or transcranial magnetic stimulation, the medical professional may focus the device on the target area in the patient's brain. For neurofeedback, the medical professional selects a neurofeedback program designed to address the patient's target brain region. For perceptual isolation, the medical professional selects the stimuli reduction method to be used. For virtual reality, the medical professional chooses a VR simulation designed to evoke a particular memory or emotion related to the patient's issue. For psychotherapy, the medical professional selects a session protocol which evokes the patient's unwanted memory or condition.

Enhancement phase 400 may also include step 409: quiesce brain activity. The patient may be placed in a resting state free from distractions to minimize brain activity in an effort to deactivate all brain regions except the default mode network of the patient.

Enhancement phase 400 may further include step 410: administering the calculated dose of the neuron-editing biologics. The neuron-editing biologics are here administered intranasally to the patient via swab or nasal inhaler. If ligand vectors are being used in this step, a medical practitioner may skip steps 411-413 and instead proceed to step 414 as described below.

Enhancement phase 400 may also include step 411: administering the brain region activator to activate the target brain regions. The medical professional administers the brain region activator method selected for the patient in Step 404.

Enhancement phase 400 may further include step 412: concentrating neural activity in the target brain region using the brain region connectome method. The brain regional connectome analysis concentrates neuronal activity in the target brain region(s) as discussed above.

Enhancement phase 400 may also include step 413: transporting the neuron-editing biologics to the active neurons within the target brain region using hemodynamics. Cerebral blood flow containing glucose, oxygen, and neuron editing biologics is transported to active neurons in the targeted area of the patient's brain, facilitating administration of the selected biologic to the appropriate area of the subject's brain.

Enhancement phase 400 may further include step 414: editing the active neurons using the neuron-editing biologics. Neuron-editing biologics transfect neurons in the target area of the patient's brain and edit the neurons' DNA or RNA to enhance activity in the region.

Enhancement phase 400 may also include step 415: enhancing the patient's cognitive capacity. Optimized brain region activity may operate to expand the patient's cognitive capacity as desired.

Enhancement phase 400 may further includes step 416: iterating steps 402-415 as needed. If multiple brain areas are indicated for treatment, the medical professional returns to Step 402 to address the next area of priority. The brain scan performed at this juncture verifies optimized brain activity in the region just treated.

Enhancement phase 400 may also include step 417: conducting post-treatment therapy. Post-treatment therapy may be used to teach/instill mind and life practices for reinforcing the gene therapy's effects into the subject by fostering supportive behavioral and epigenetic factors. This therapy can employ psychological counseling protocols such as cognitive behavioral therapy, mindfulness-based stress reduction, neuro-linguistic programming, meditation, visualization and other techniques.

Table 2 illustrates the practical application of the enhancement 400 in treating thirteen examples of common cognitive enhancements:

TABLE 2
practical application of the enhancement 400 in treating
thirteen examples of common cognitive enhancements
Enhancement

| | | | | | | | |
|---|---|---|---|---|---|---|---|
| | | Transcranial | Transcranial | | Perceptual | Virtual | |
| Objective | Brain Regions | ultrasound | magnetic stim. | Neurofeedback | isolation | reality | Psychotherapy |

| | | | | | |
|---|---|---|---|---|---|
| 1. Raise conscious awareness | Posterior | ● | ● | | |
| 2. Decrease inattention | cingulate cortex | ● | ● | | |
| 3. Reduce mind-wandering | (ventral) | ● | ● | | |
| 4. Lower craving | | ● | ● | | |
| 5. Sharpen mental focus | | ● | ● | | |
| 6. Increase concentration | | ● | ● | | |
| 7. Enhance mindfulness | | ● | ● | | |
| 8. Improve meditation | | ● | ● | | |
| 9. Increase intuition | | ● | ● | | |
| 10. Improve ESP | Caudate | ● | | | |
| 11. Cognitive therapy for | Various | | | | ● |
| unwanted behaviors | | | | | |
| 12. Cognitive therapy for | Various | | | ● | ● |
| desired behaviors | | | | | |
| 13. Alleviate cognitive | Posterior | ● | ● | | |
| impairment symptoms | cingulate cortex | | | | |

Table 2 lists several exemplary practical applications for achieving cognitive enhancement. Several of these such as objectives 1-9 include general-purpose cognitive enhancements: raising conscious awareness, decreasing inattention, lowering craving, sharpening mental focus, increasing concentration, and improving meditation. These cognitive enhancements 1-9 may be achieved by administering neuron-editing biologics for decreasing neuron excitability, along with transcranial pulsed ultrasound and/or perceptual isolation brain region activation techniques which direct the biologics to the ventral posterior cingulate cortex (PCC) via hemodynamic vectoring. Reduced PCC activity has been experimentally correlated with increased attention, conscious awareness, mental acuity, clarity, focus, concentration, and mindfulness, and with reduced mind-wandering, inattention and cravings.

Regarding cognitive enhancement 10 listed in Table 2, studies have shown that individuals who can demonstrate extra-sensory perception (ESP). Patients who desire to raise their ESP abilities may benefit from administering neuron-editing biologics for increasing neuron excitability, along with transcranial pulsed ultrasound brain region activation techniques which direct the biologics to specific brain regions via hemodynamic vectoring. NB the ‘caudate’ region noted in the unpublished pilot study is actually the internal capsule/striatal bridges region

Regarding cognitive enhancement 11 listed in Table 2, psychotherapy session protocols can be designed to stimulate the underlying causes of the patient's unwanted behavior, including attitudes, emotions, beliefs, expectations, and memories. This mental activity will generate brainwave and neural activity in corresponding areas in the patient's brain. Neuron editing biologics administered for reducing neuron excitability may then be transported to these active areas via hemodynamic vectoring.

Regarding cognitive enhancement 12 listed in Table 2, psychotherapy session protocols and immersive multi-sensory VR programs can be designed to help the patient visualize and experience desired behaviors, including attitudes, feelings, beliefs, expectations, and assumptions. This mental activity will generate neural activity in corresponding areas in the patient's brain. Neuron editing biologics administered for altering neuron excitability will be transported to these active areas via hemodynamic vectoring.

Regarding cognitive enhancement 13 listed in Table 2, mild cognitive impairment (MCI) may affect many different regions of the brain. It can be treated indirectly by administering a cognitive enhancement protocol to offset its effects. This protocol involves administering neuron-editing biologics for decreasing neuron excitability in the posterior cingulate cortex (PCC), in conjunction with transcranial pulsed ultrasound or perceptual isolation techniques which direct the biologics to the PCC via hemodynamic vectoring.

Various aspects of this disclosure relate to a method to treat symptoms of anxiety in a subject, comprising providing a first AAV vector and a second AAV vector and administering the first AAV vector and the second AAV vector to the subject, wherein the first AAV vector is an AAV9 vector; the first AAV vector comprises a first nucleic acid that comprises a first nucleotide sequence that comprises a nuclease-encoding sequence that encodes a nuclease; the nuclease is a Cas9 nuclease that is spCas9; the first nucleotide sequence encodes a first promoter that is operably linked to the first nucleotide sequence such that the first promoter can mediate transcription of the nuclease-encoding sequence in neurons; the first promoter is a human methyl CpG binding protein 2 (MECP2) promoter; the second AAV vector is an AAV9 vector; the second AAV vector comprises a second nucleic acid that comprises a second nucleotide sequence that encodes a gRNA; the second nucleotide sequence comprises a targeting sequence and a nuclease-recruiting sequence; the targeting sequence is complementary to a genomic nucleotide sequence; either the genomic nucleotide sequence or a reverse complement of the genomic nucleotide sequence encodes a portion of human 5HT-2A receptor; either the genomic nucleotide sequence or the reverse complement is a portion of a second exon that encodes the human 5HT-2A receptor; the targeting sequence comprises at least 90 percent sequence identity with at least 10 consecutive nucleotides set forth in GAUUCUGGAUGGCGACGUAG (SEQ ID NO. 3); the nuclease-recruiting sequence is configured to recruit the nuclease; the second nucleotide sequence encodes a second promoter that is operably linked to the targeting sequence and the nuclease-recruiting sequence such that the second promoter can mediate transcription of the targeting sequence and the nuclease-recruiting sequence in neurons; the second promoter is a human U6 promoter; the first AAV vector is administered intranasally; at least 200 billion particles of the first AAV vector are administered to the subject; the first AAV vector is administered in an amount sufficient to achieve a MOI per target cell of at least 3; the second AAV vector is administered intranasally; at least 200 billion particles of the second AAV vector are administered to the subject; the second AAV vector is administered in an amount sufficient to achieve a MOI per target cell of at least 3; the administering results in a frameshift mutation in the second exon of human 5HT-2A receptor; the frameshift mutation results in a premature stop codon that inhibits translation of a third exon of human 5HT-2A receptor such that the translation results in a non-functional human 5HT-2A receptor; the first AAV vector and the second AAV vector are administered in amounts sufficient to reduce expression of functional human 5HT-2A receptor in the brain of the subject by at least 20 percent; and reduced expression of functional human 5HT-2A receptor in the brain of the subject treats the symptoms of anxiety.

The method of the preceding paragraph sets forth a specific embodiment, but many different variations of this specific embodiment fall within the scope of the inventive compositions and methods of this disclosure. For example, other viral vectors display a tropism for neurons are viable including lentiviral vectors and other AAV serotypes such as serotype 8, and such viral vectors may be used instead of AAV9. Different nucleases may alternatively be used including TALENS, zinc-finger nucleases, and meganucleases. Different promoters may alternatively be used such as the synapsin I promoter. Different delivery methods may be used such as, for example, injection. Different numbers of viral particles may be administered, for example, depending on the efficiency of the knockout strategy and the desired efficiency of the method. Additionally, the subject might present with a different condition that is treatable by knocking out 5HT-2A receptor such as depression. These variations and others fall within the scope on this disclosure, the paragraphs that follow, and various patent claims. The scope of any patent claim that matures from this disclosure shall not be construed as requiring any specific feature that is not explicitly recited in the claim based on the disclosure of specific embodiments in the preceding paragraph or elsewhere in this specification.

Various aspects of this disclosure relate to a method to modulate brain activity in a brain of a subject, comprising: providing a first AAV vector and a second AAV vector; and administering the first AAV vector and the second AAV vector to the brain of the subject, wherein: the first AAV vector comprises a first nucleic acid that comprises a first nucleotide sequence that comprises a nuclease-encoding sequence that encodes a nuclease; the second AAV vector comprises a second nucleic acid that comprises a second nucleotide sequence that encodes a gRNA; the second nucleotide sequence comprises a targeting sequence and a nuclease-recruiting sequence; the targeting sequence is complementary to a genomic nucleotide sequence that encodes a portion of a protein; the nuclease-recruiting sequence is configured to recruit the nuclease; and the administering results in a mutation that affects translation of the protein in the brain, which modulates brain activity in the subject.

The phrase “recruit the nuclease” refers to an interaction between a gRNA and the nuclease, which brings the nuclease into proximity with a portion of genomic DNA of the subject, which gRNA is designed such that a portion of the gRNA is complementary to the portion of genomic DNA and therefore results in a base-pairing interaction between the portion of the gRNA and the portion of genomic DNA to thereby enable the nuclease to cleave a phosphodiester bond in or adjacent to the portion of genomic DNA.

The term “a mutation that affects translation” includes any mutation that decreases translation of a fully-functional protein and includes frameshift and insertion/deletion (INDEL) mutations that result in one or more premature stop codons that result, for example, in translation of a truncated version of the protein. In some specific embodiments, “a mutation that affects translation” refers to a frameshift or INDEL mutation that results in one or more premature stop codons that inhibit the translation of the C-terminus of a G protein-coupled receptor. In some very specific embodiments, “a mutation that affects translation” refers to a frameshift or INDEL mutation that results in one or more premature stop codons that inhibit the translation of the C-terminus of a G protein-coupled receptor such that either the G protein-coupled receptor is not trafficked to the neuron cell membrane and/or the G protein-coupled receptor is no longer capable of modulating G protein-mediated signaling cascades and/or the G protein-coupled receptor no longer couples to G proteins.

In some embodiments, the first AAV vector is an AAV9 vector. In some embodiments, the second AAV vector is an AAV9 vector. In some specific embodiments, the first AAV vector is an AAV9 vector, and the second AAV vector is an AAV9 vector.

In some embodiments, the nuclease is a Cas9 nuclease. In some specific embodiments, the nuclease is a Cas9 nuclease, and the Cas9 nuclease is spCas9.

In some embodiments, the protein is a G protein-coupled receptor.

In some embodiments, the protein is a G protein-coupled receptor, and binding of the G protein-coupled receptor to a neurotransmitter activates phospholipase C to cause calcium release. In some specific embodiments, the protein is a G protein-coupled receptor; binding of the G protein-coupled receptor to a neurotransmitter activates phospholipase C to cause calcium release; and the administering modulates brain activity in the subject by modulating neurotransmitter-dependent calcium release in neurons that contain the mutation.

In some embodiments, the protein is 5HT-2A receptor.

In some embodiments, the protein is a G protein-coupled receptor, and binding of the G protein-coupled receptor to a neurotransmitter inhibits adenylyl cyclase to decrease CAMP. In some specific embodiments, the protein is a G protein-coupled receptor; binding of the G protein-coupled receptor to a neurotransmitter inhibits adenylyl cyclase to decrease cAMP; and the administering modulates brain activity in the subject by modulating neurotransmitter-dependent cAMP concentration in neurons that contain the mutation.

In some embodiments, the protein is 5-hydroxytryptamine receptor 1A (5HT-1A receptor).

In some embodiments, the mutation introduces a premature stop codon into a gene that encodes the protein. In some specific embodiments, the mutation introduces a premature stop codon into a gene that encodes the protein, and the premature stop codon inhibits the expression of at least one exon of the gene. In some very specific embodiments, the mutation introduces a premature stop codon into a gene that encodes the protein; the premature stop codon inhibits the expression of at least one exon of the gene; and the at least one exon encodes a C-terminal intracellular region of the G protein-coupled receptor.

In some embodiments, the targeting sequence comprises at least 90 percent sequence identity with at least 10 consecutive nucleotides set forth in SEQ ID NO. 3. In some specific embodiments, the targeting sequence comprises at least 100 percent sequence identity with at least 10 consecutive nucleotides set forth in SEQ ID NO. 3. In some specific embodiments, the targeting sequence comprises at least 90 percent sequence identity with at least 15 consecutive nucleotides set forth in SEQ ID NO. 3. In some even more specific embodiments, the targeting sequence comprises at least 100 percent sequence identity with at least 15 consecutive nucleotides set forth in SEQ ID NO. 3.

In some embodiments, the gRNA has at least 30 percent efficiency at causing the mutation in human neurons. In some specific embodiments, the gRNA has at least 45 percent efficiency at causing the mutation in human neurons. In some very specific embodiments, the gRNA has at least 60 percent efficiency at causing the mutation in human neurons.

In some embodiments, the targeting sequence is SEQ ID NO. 3. In some specific embodiments, the targeting sequence is SEQ ID NO. 3, and the gRNA has at least 60 percent efficiency at causing the mutation in human neurons.

In some embodiments, the method comprises administering the first AAV vector in an amount sufficient to achieve a MOI per target cell of at least 1. In some specific embodiments, the method comprises administering the first AAV vector in an amount sufficient to achieve a MOI per target cell of at least 3. In some very specific embodiments, the method comprises administering the first AAV vector in an amount sufficient to achieve a MOI per target cell of at least 5.

In some embodiments, the method comprises administering the second AAV vector in an amount sufficient to achieve a MOI per target cell of at least 1. In some specific embodiments, the method comprises administering the second AAV vector in an amount sufficient to achieve a MOI per target cell of at least 3. In some very specific embodiments, the method comprises administering the second AAV vector in an amount sufficient to achieve a MOI per target cell of at least 5.

In some embodiments, the first AAV vector is administered intranasally. In some embodiments, the second AAV vector is administered intranasally. In some specific embodiments, the first AAV vector and the second AAV vector are administered intranasally.

In some embodiments, the method comprises administering an olfactory test to the subject prior to administering the first AAV vector and the second AAV vector, wherein one or both of the first AAV vector and the second AAV vector are administered intranasally. In some specific embodiments, the method comprises administering an olfactory test to the subject prior to administering the first AAV vector and the second AAV vector and adjusting the number of particles of the first AAV vector and/or second AAV vector that are administered based on the results of the olfactory test, wherein one or both of the first AAV vector and the second AAV vector are administered intranasally; more particles of the first AAV vector and/or second AAV vector are administered if the olfactory test indicates that the cribiform plate and adjacent olfactory epithelium of the subject are less permeable to transit of biologics from the intranasal cavity into the olfactory bulb and adjacent regions of the forebrain; and less particles of the first AAV vector and/or second AAV vector are administered if the olfactory test indicates that the cribiform plate and adjacent olfactory epithelium of the subject are more permeable to transit of biologics from the intranasal cavity into the olfactory bulb and adjacent regions of the forebrain. In some very specific embodiments, the method comprises administering an olfactory test to the subject prior to administering the first AAV vector and the second AAV vector and adjusting the number of particles of the first AAV vector and/or second AAV vector that are administered based on the results of the olfactory test, wherein one or both of the first AAV vector and the second AAV vector are administered intranasally; more particles of the first AAV vector and/or second AAV vector are administered if the olfactory test indicates that the subject has a poor sense of smell because a poor sense of smell indicates that the cribiform plate and adjacent olfactory epithelium of the subject are less permeable to transit of biologics from the intranasal cavity into the olfactory bulb and adjacent regions of the forebrain; and less particles of the first AAV vector and/or second AAV vector are administered if the olfactory test indicates that the subject has a normal sense of smell because a normal sense of smell indicates that the cribiform plate and adjacent olfactory epithelium of the subject are more permeable to transit of biologics from the intranasal cavity into the olfactory bulb and adjacent regions of the forebrain.

In some embodiments, the method comprises focusing pulsed ultrasound in regions of the brain, wherein the regions of the brain in which the pulsed ultrasound is focused display an increased prevalence of mutation per neuron relative to other regions of the brain in which the pulsed ultrasound is not focused. Transcranial pulsed ultrasound can be configured to activate neurons in target regions of the brain, which activity increases blood flow to the target regions, which increases the flow of therapeutics (such as the first AAV vector and the second AAV vector) to the target regions.

In some embodiments, the method comprises focusing repetitive Transcranial Magnetic Stimulation in regions of the brain, wherein the regions of the brain in which the repetitive Transcranial Magnetic Stimulation is focused display an increased prevalence of mutation per neuron relative to other regions of the brain in which the repetitive Transcranial Magnetic Stimulation is not focused. Transcranial Magnetic Stimulation can be configured to activate neurons in target regions of the brain, which activity increases blood flow to the target regions, which increases the flow of therapeutics (such as the first AAV vector and the second AAV vector) to the target regions.

In some embodiments, the region comprises at least a portion of one or more of the amygdala, dorsal amygdala, hippocampus, hypothalamus, interpeduncular nucleus, anterior cingulate cortex, dorsal anterior cingulate cortex, posterior cingulate cortex, prefrontal cortex, ventromedial prefrontal cortex, pons, pontine nuclei, cerebellum, Purkinje layer of the cerebellum, orbital gyrus, and caudate nucleus. In some specific embodiments, the region comprises one or more of the olfactory bulb, cortex, subcortical regions, cerebellum, Purkinje cell layer of the cerebellum, pons, pontine gray nuclei, interpeduncular nucleus, thalamus, hypothalamus, hippocampus, and amygdala. In some very specific embodiments, the region comprises one or more of the olfactory bulb, cortex, cerebellum, Purkinje cell layer of the cerebellum, pons, pontine gray nuclei, and interpeduncular nucleus.

Various aspects of this disclosure relate to a method to treat a mental health condition in a subject, comprising determining that the subject presents with the mental health condition and performing a method as described anywhere in this disclosure.

In some embodiments, the mental health condition is stress, anxiety, ADD, OCD, PTSD, bipolar disorder, a personality disorder, memory concern, dementia, or inattention. In some specific embodiments, the mental health condition is anxiety or a symptom of the mental health condition is anxiety. In some very specific embodiments, the mental health condition is anxiety.

Various aspects of this disclosure relate to a method to reduce body weight in a subject, comprising performing a method as described anywhere in this disclosure. In some embodiments, the method is effective at reducing body fat in the subject.

Various aspects of this disclosure relate to a recombinant nucleic acid, comprising a targeting sequence that comprises at least 90 percent sequence identity with at least 10 consecutive nucleotides set forth in SEQ ID NO. 3 or a reverse complement thereof. In some specific embodiments, the targeting sequence comprises at least 100 percent sequence identity with at least 10 consecutive nucleotides set forth in SEQ ID NO. 3. In some specific embodiments, the targeting sequence comprises at least 90 percent sequence identity with at least 15 consecutive nucleotides set forth in SEQ ID NO. 3. In some even more specific embodiments, the targeting sequence comprises at least 100 percent sequence identity with at least 15 consecutive nucleotides set forth in SEQ ID NO. 3. In some very specific embodiments, the targeting sequence is SEQ ID NO. 3.

The human HTR2A gene encodes SEQ ID NO. 3. In some embodiments, the recombinant nucleic acid comprises a promoter that is operably linked to SEQ ID NO.3, and the promoter is not the HTR2A promoter. In some specific embodiments, the recombinant nucleic acid comprises a human U6 promoter or a variant thereof. The human U6 promoter and optimized variants thereof are commonly used to drive transcription of gRNA in many different cell types.

In some embodiments, the recombinant nucleic acid comprises a nuclease-recruiting sequence. In some specific embodiments, the recombinant nucleic acid comprises a Cas9 nuclease-recruiting sequence. In some very specific embodiments, the recombinant nucleic acid comprises a trans-activating CRISPR RNA (tracrRNA) sequence. The tracrRNA sequence specifically interacts with Cas9 nuclease.

In some embodiments, the recombinant nucleic acid comprises inverted terminal repeats (ITRs). In some specific embodiments, the recombinant nucleic acid comprises 2 ITRs that are configured to package the recombinant nucleic acid in an AAV vector.

In some embodiments, the recombinant nucleic acid is a plasmid. In some specific embodiments, the recombinant nucleic acid is a plasmid that comprises an origin of replication. In some very specific embodiments, the recombinant nucleic acid is a plasmid that comprises a bacterial origin of replication.

In some embodiments, the recombinant nucleic acid comprises an antibiotic resistance gene. In some specific embodiments, the recombinant nucleic acid comprises an ampicillin resistance gene.

Various aspects of this disclosure relate to an AAV vector comprising a recombinant nucleic acid as described herein. In some embodiments, the AAV vector is an AAV9 vector.

Various aspects of this disclosure relate to a cell line comprising a recombinant nucleic acid as described herein. In some embodiments, the cell line is a human cell line. In some specific embodiments, the cell line comprises HEK293 cells.

Various aspects of this disclosure relate to a composition comprising a AAV vector as described herein and cesium chloride.

### Example 1: Design of Guide RNA to Knockout HTR2A in Mouse Neurons

The HTR2A mouse gene encodes a single protein-coding transcript HTR2A-201. Two in silico strategies were designed to knockout the gene with both strategies utilizing the same cut site for Cas9 in the first coding exon. In the first strategy, a Cas9-directed double-strand break near the start site (ATG) would be repaired by non-homologous end joining (NHEJ), introducing random INDELs and potential frameshift mutations. In the second strategy, CRISPR/Cas9 would cut the first coding exon, and homology-directed repair (HDR) systems would lead to insertion of a STOP-pA cassette to terminate transcription of the HTR2A gene. The targeting sequence chosen in both cases, TGCAATTAGGTGACGACTCGAGG (SEQ ID NO: 1), would give no predicted off-target cut sites, produce an 86.6% frameshift frequency, and have a precision score of 0.55. An additional 200 base pair (bp) knock-in STOP-pA donor cassette was manufactured that would insert two stop codons, which are underlined below:

(SEQ ID NO: 2)
GAAGACAATATCTCCCTGAGCTCAATTCCAAACTCCTTAATGCAATTAG
GTGACTAGTGAATTCAACTTGTTTATTGCAGCTTATAATGGTTACAAAT
AAAGCAATAGCATCACAAATTTCACAAATAAAGCATTTTTTTCACTGCT
CGAGGCTCTACCCTAATGACTTCAACTCCAGGGATGCTAACACTTCCGA
AGCC

The 200 bp knock-in STOP-pA donor cassette (SS ODN template) requires HDR, a process that mainly occurs in the S and G2 phases of the cell cycle and is thought to rarely take place in mature neurons. If HDR-mediated insertion of the SS ODN sequence does not occur, it is still possible to produce potential genomic changes in postmitotic neurons by random INDELs induced by Cas9 cleavage and non-homologous end-joining (NHEJ) repair introducing premature stop codons. Thus, the strategy ensured potential gene editing within neurons regardless of whether HDR or NHEJ were the predominant repair systems.

### Example 2: Confirmation of Knockout Strategy In Vitro

To evaluate the activity of spCas9 using the above gRNA design, T7E1 assays were performed. These assays detect heteroduplex DNA that results from annealing DNA strands that have been modified after a gRNA/Cas9 mediated cut to DNA strands. Briefly, following preparation of genomic DNA, amplification of the target site by PCR was performed using designed primers on either side of the target sequence. Following amplification, T7E1 digests were performed to detect potential mismatches at the target site.

To test the knockout strategy both in vitro and in vivo, experiments were performed in mouse wild-type embryonic stem cells (ESCs). Naïve mouse ESCs were transfected with gRNA and Cas9, and cells were selected with puromycin.

Cleavage efficiency was determined by digesting annealed PCR products with T7E1, and fragments were analyzed following gel electrophoresis. The gRNA targeted Cas9 to generate double-strand breaks, producing the expected size products of 196 and 179 bp following cleavage of the predicted 375 base pair amplicon. Overall, gRNA cleavage efficiency was low. However, because both NHEJ and HDR-based editing possibilities were possible, a balance approach was taken, thus efficiency is less relevant than the placement of the cleavage site with respect to the homology arms. In other words, HDR efficiency does not linearly correlate with Cas9 cutting efficiency.

Confirmation of insertion of the SS ODN stop template following HDR repair was identified in F0 founder mice following injection of gene edited ESCs cells into embryos and transfer to surrogate female mice. F0 offspring demonstrated the correct insertion of the SS ODN sequence containing the two premature stop codons.

### Example 3. Design of Adeno-Associated Viral Vector Platform

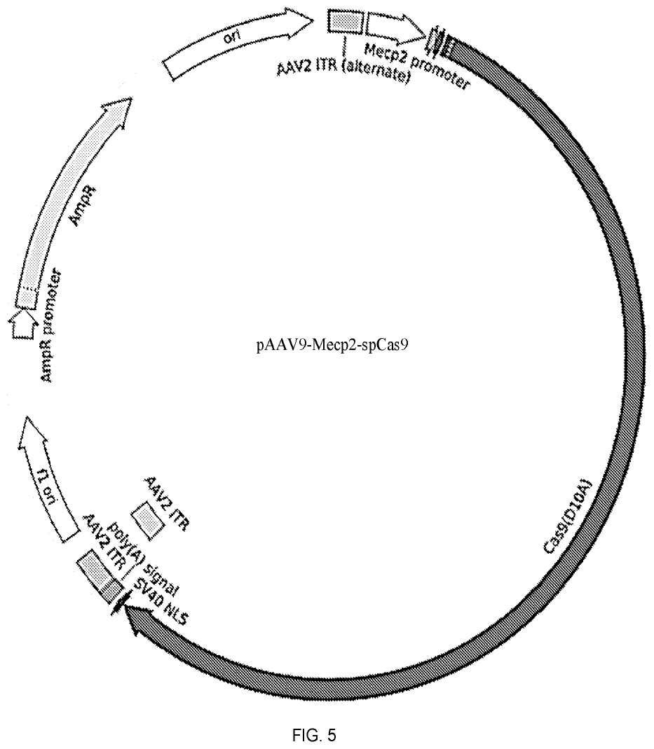

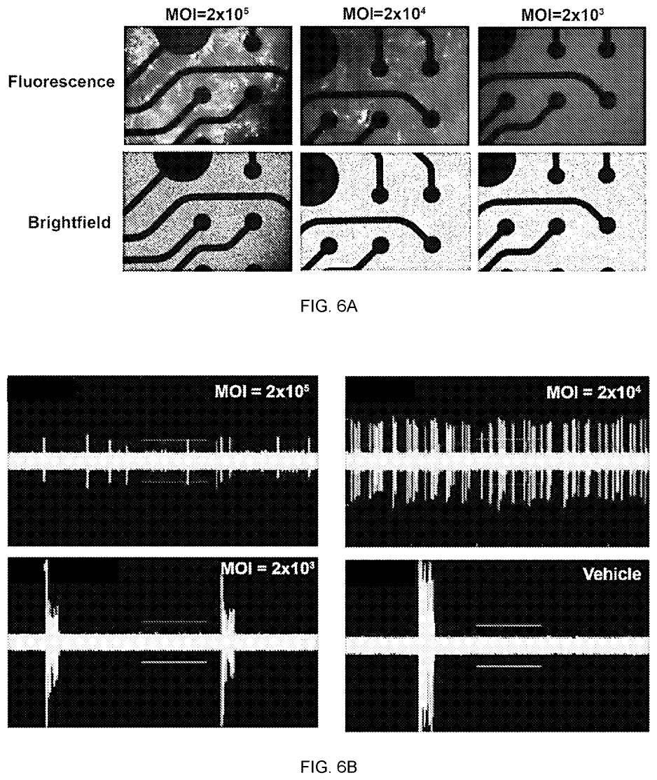

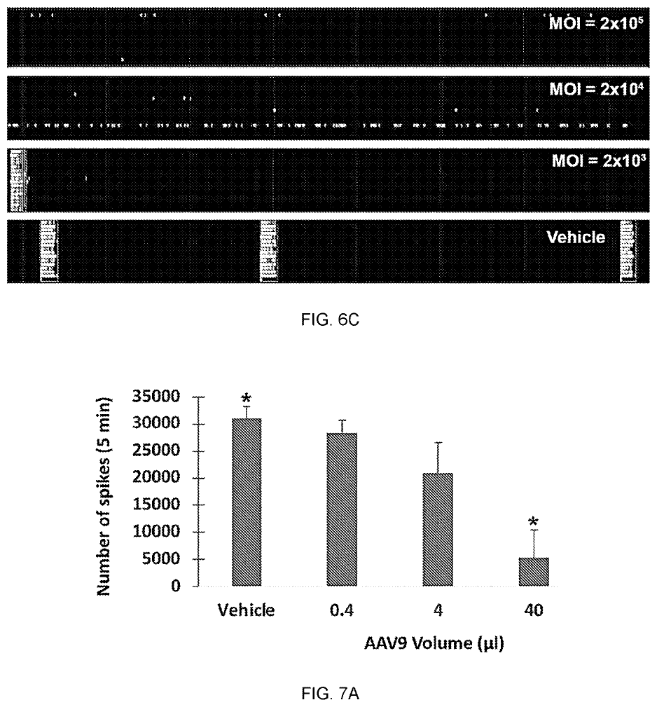

Two different adeno-associated virus serotype 9 (AAV9) vectors were designed to deliver spCas9 and gRNA to CNS neurons. The design of two vectors was necessary based on the limited carrying capacity of 4.7 kb for AA9 viruses. Dual AAV9 systems were created to expand the capacity. The first AAV9 expressed spCas9 under a neuronal-specific promoter, MeCP2 (~4.2 kb), and the spCas9 vector utilized the PX551 plasmid from Addgene (pAAV9-Mecp2-spCas9; FIG. 5). The second AAV9 vector consisted of the gRNA sequence, 200-bp SS ODN donor template, and a green-fluorescence protein (GFP) reporter under the U6 promoter (AAV9-gRNA-U6-GFP). Large-scale transfection of plasmids was carried out in HEK293 cells and purified through a series of cesium chloride centrifugations. Titer load (in genome copy number per ml, or GC/ml) was determined by quantitative real-time PCR, with typical yields of 20 trillion GC/ml. Both AAV9 vectors were stored in PBS with 5% glycerol at −80 °C until used.

### Example 4. Confirmation of AAV Vector Strategy In Vitro

24-well MEA plates were coated with 500 μl 0.07% polyethyleneimine and incubated for 1 hour. Plates were then washed 4 times in sterile deionized water and dried overnight in a biosafety cabinet. Primary cortical neurons from fresh mouse brain embryos were isolated and plated onto coated 24-well plates at a density of 500,000 cells/well. Cortical neurons were maintained in Neurobasal™-A Medium, supplemented with B27, GlutaMAX™, and antibiotics (100 U/ml penicillin, and 100 μg/ml streptomycin). Cultured neurons were incubated at 37 °C and 5% CO₂, and half of the media was exchanged with fresh, complete media every three days. The AAV-treated cocktail consisted of an equal mixture of AAV9-gRNA-U6-GFP and AAV9-Mecp2-spCas9 suspended in 0.9% NaCl (saline). The final concentration of each AAV9 vector in this mixture was 2.5 trillion GC/ml. Infection of cortical neurons was based on the MOI, which refers to the number of viral particles per cell. Three different MOI concentrations were used: 200,000 (which is equivalent to a 100 billion total viral particles), 20,000, and 2,000. Control neurons received vehicle (40 μl of saline) an equivalent to the volume of fluid given at MOI of 200,000. Treatment was performed in triplicate. Treatment occurred on Day 6 for 24 hours at 37 °C, at which time the media was replaced with fresh, complete media. MEA analysis was then performed on Day 14.

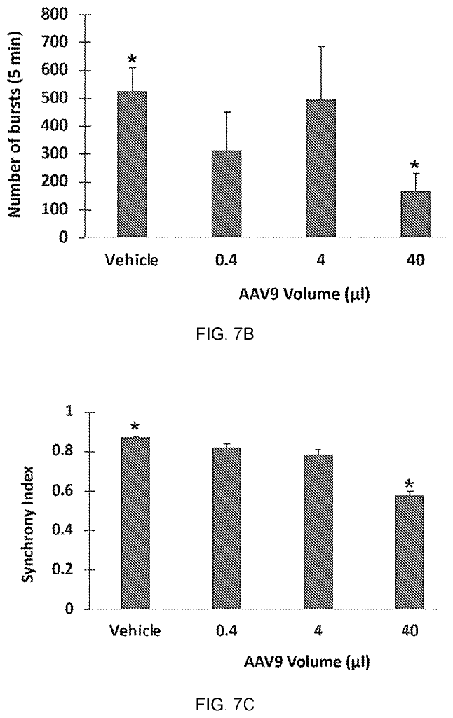

A concentration-dependent effect on MEA parameters was observed in neurons that received the AAV cocktail including a significant decrease in the total number of spikes, the number of bursts, and in the synchrony index (FIG. 7A-7C). Importantly, there was no evidence of toxicity at any concentration following treatment, and neurons were healthy with no rupture of neurites. Taken together, these data support the conclusion that the AAV9 cocktail is non-toxic and decreases the collective electrical activity of cortical neuron networks in vitro.

### Example 5. Administration of AAV Vectors to Mice

4-5 weeks old male CD-1 mice were group-housed 5 or 6 per cage in a temperature-controlled room (21-22 °C) with a light-dark cycle (12 h/12h; lights on: 17:30-05:30; lights off: 05:30-17:30) and with food and water available ad libitum. Mice were acclimated to the animal facility for at least 1-week before beginning experimental procedures.

The AAV-treated group received an equal mixture of AAV9-gRNA-U6-GFP and AAV9-Mecp2-spCas9 suspended in 0.9% NaCl (saline). The concentration of AAV9 stock mixture was ~5 trillion GC/ml. For each behavioral test described below, a new, independent batch of AAV9 vectors was prepared. The AAV cocktail was administered twice on day one (morning and afternoon) five weeks before the behavioral tests. For intranasal delivery of AVV, mice were hand-restrained with the nose positioned to facilitate the dosing. A meniscus of AAV solution droplet (10 μl per nostril) was then formed at the tip of the micropipette and presented for inhalation in each of the nares of the mouse. Each mouse received a total of 40 μl of AAVs equivalent to ~200 billion viral particles. Vehicle-treated animals received 40 μL of saline for each treatment following the same protocol.

### Example 6. Verification of Knockout by qPCR

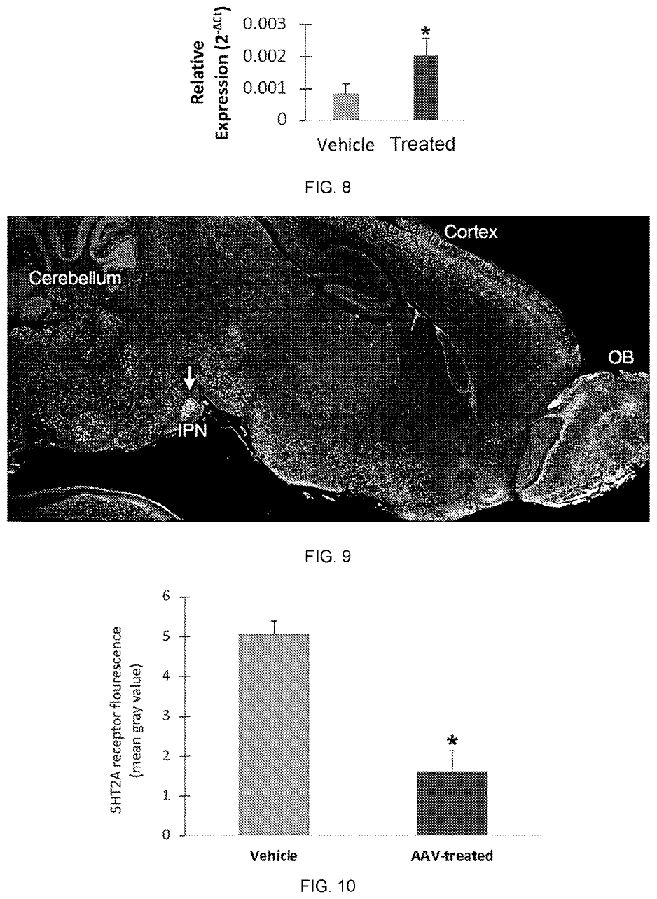

Following treatment of mice on day one, mice were sacrificed five weeks post intranasal treatment, and brains were snap-frozen. Total RNA was extracted from vehicle control (N=5) and AAV-treated mice (N=5) using a standard extraction protocol with TRIzol™ and then dissolved in diethyl pyrocarbonate-treated deionized water and quantified. Following reverse transcription, qPCR was performed. Treatment led to a significant 8.47-fold decrease in HTR2A expression (p=0.05) (FIG. 8).

To analyze gene editing, whole brain (N=5) or olfactory bulb (N=5) genomic DNA was analyzed by next-generation sequencing (NGS) around the predicted Cas9 cleavage site of the HTR2A gene. An INDEL frequency of 0.07-0.13% was detected in all 5 brain samples. In all brain cases examined, the INDEL profile revealed single base pair deletions of adenine at identical positions near the PAM site. Other INDELs observed were single base deletions in cytosine at the same position in 3 out of 4 brain cases and in guanine in one brain sample. A similar result was obtained in the olfactory bulb samples from the same five animals.

For the single base pair deletions in adenine, these two INDELs would result in a predicted frame-shift mutation leading to the insertion of premature stop codons. It is noteworthy that none of the INDELs revealed an insertion of the 200 bp donor SS ODN repair template, suggesting that NHEJ is the primary method of repair in generation of nonsense mutations. The low frequency of editing is unsurprising given the large dilution effect of brain genomic DNA. Neurons make up less than 10% of the total number of cells in the brain, and only a subpopulation of these neurons would in fact be targeted by the AAV cargo. Taken together, these results support CRISPR-mediated gene editing of HTR2A in vivo that is most likely occurring through the single-nucleotide INDEL formation and generation of premature stop codons via NHEJ.

### Example 7. Verification of mRNA Knockout by Microscopy

Five weeks following intranasal AAV9 delivery of co-packaged Cas9 DNA and a HTR2A-targeting gRNA, immunofluorescence studies were conducted in fixed, sagittal mouse sections to examine 5HT-2A receptor protein expression. Mice were anesthetized with 5% isoflurane/oxygen mixture and sacrificed by decapitation. Brains with olfactory bulbs were extracted and fixed in 4% formalin for 48 hours and transferred to vials containing 1% formalin in PBS buffer.

gRNA expression was visualized by GFP fluorescence while 5HT-2A receptor labeling was accomplished utilizing an antibody previously demonstrated to be highly specific for the mouse 5HT-2A receptor. Following dehydration, 4 μm paraffin-embedded, sagittal sections were cut lateral to the midline and used for immunofluorescence labeling. Briefly, all tissue sections were labeled with anti-GFP antibody (rabbit mAB #2956) 1:1,000 (Cell Signaling Technology, Inc., Danvers, MA, USA) or anti-5HT-2A receptor antibody (rabbit polyclonal, #24288) at 1:500 dilution (Immunostar, Hudson, WI). Secondary antibodies were conjugated to FITC or Cy3. DAPI was used as a nuclear stain. Whole slide scanning was performed using a Pannoramic Midi II scanner using a 40× objective lens with optical magnification of 98×, 0.1 μm/pixel.

The level of 5HT-2A receptor fluorescence was quantified using ImageJ software. This was accomplished by capturing 50× immunofluorescence images from three different fields (cortex, cerebellum, and subcortical regions) in three separate tissue sections from each group (vehicle control or AAV-treated). All data were expressed as the mean gray value±SEM. The mean gray value reflects the sum of the gray values of all the pixels in the selected area divided by the number of pixels. The area of selection in square pixels was not significantly different between vehicle-controls (676,181) and AAV-treated (671,647), p=0.639, N=9 for each group.

Intranasal administration of the AAV9 vectors (final concentration of mixture was ~200 billion viral particles) resulted in widespread expression of GFP/gRNA throughout the CNS including olfactory bulb, cortex and sub-cortical areas including the interpeduncular nucleus (IPN) (FIG. 9), a major connectome for stress-mediated pathways. In AAV-treated mice, GFP (gRNA) labeling was strongest in the cell bodies of neurons, and, when colocalized with 5HT-2A in AAV-treated mice, staining of 5HT-2A appeared weaker in the cell bodies with some labeling still apparent within apical dendrites. As expected, there was little evidence of GFP (gRNA) staining in vehicle-control mice.

ImageJ quantification indicated a significant 68% decrease in 5HT-2A receptor fluorescence of AAV-treated mice as compared to vehicle controls (FIG. 10), p=0.0026. These results can be interpreted to suggest that treatment of the co-packaged spCas9 DNA and a HTR2A-targeting gRNA led to a decrease in 5HT-2A receptor staining. Co-localization of gRNA/GFP together with 5HT-2A receptor fluorescence was evident throughout the CNS including layers I-III of the cortex, the Purkinje cell layer of the cerebellum, and subcortical areas including within pontine gray nuclei (FIG. 11). While strong 5HT-2A receptor labeling was evident in vehicle controls in these regions, in AAV-treated mice staining appeared to be less robust. Importantly, knockout of the 5HT-2A occurred in brain regions linked to anxiety-mediated pathways including the interpeduncular nucleus and pontine gray nuclei.

### Example 8. Marble Burying Test

The marble burying test is used to record the number of marbles buried by mice placed in a novel environment. The theoretical basis for marble burying as an anxiety-related behavior stems from findings that anxiolytics and selective serotonin reuptake inhibitors reduce this behavior, and from the well-documented innate response of mice to bury threating objects.

The marble-burying test apparatus consisted of transparent polycarbonate cages (30 cm×18 cm×19 cm) containing a 5 cm layer of fine sawdust bedding and 20 glass marbles (diameter: 1.5 cm) spaced evenly along the walls of the cage. Each animal was placed individually in the cage where it remained for a 20-minute test session. On termination of the test session, the animals were removed from the cage, and the number of marbles with at least two-thirds of the marbles buried in the sawdust was counted. Anxious mice will bury more marbles.

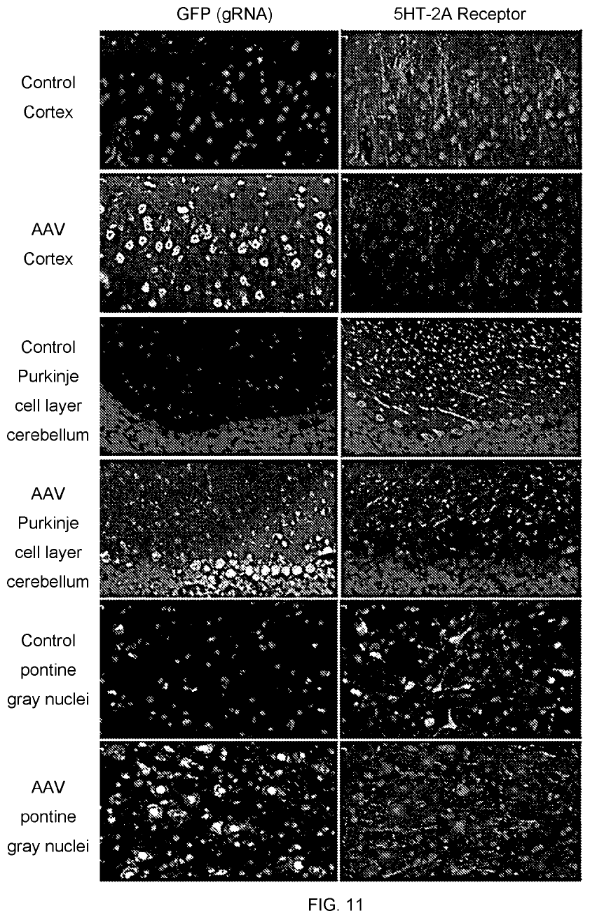

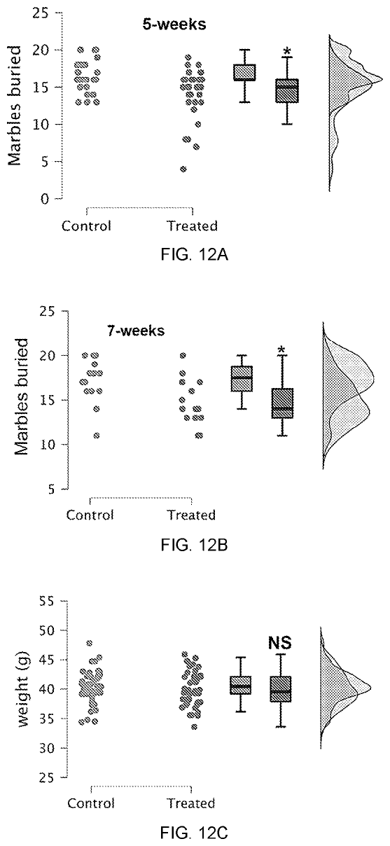

Results for 5-week AAV-treated mice indicated a 14.8% decrease in the number of marbles buried compared to vehicle-controls (N=30, p=0.0008) (FIG. 12A). Similar results were obtained following a doubling of the dose at 7-weeks post treatment (N=15, p=0.013) (FIG. 12B). Following treatment of mice with co-packaged Cas9 DNA and gRNA on day 1, there were no reported observational differences in mouse behavior, and treated mice did not differ significantly in weight at the end of the 5-week trial (p=0.569), (FIG. 12C) suggesting no major off-target edits in critical genes occurred.

### Example 9. Light-Dark Behavioral Test

The light/dark box test is a well-characterized method for evaluating the relative anxiety status of mice. The light/dark paradigm in mice is based on a conflict between the innate aversion to brightly illuminated areas and spontaneous exploratory activity. If given a choice, mice prefer the dark box. Anxiolytic compounds increase the total duration of time spent in the lit area as well as the number of entries into the lit box area.

Mice were given a choice between exploring a brightly lit chamber or a dark chamber; anxious mice will spend more time in the dark chamber. The light/dark box apparatus consisted of two PVC (polyvinylchloride) boxes (19× 19×15 cm) covered with plexiglass. One of these boxes was opaque and not illuminated. The other box was illuminated by desk lamp placed above and providing approximately 2000 Lux. An opaque plastic tunnel (5× 7× 10 cm) separated the dark box from the illuminated one. A camera linked to a video tracking system (Viewpoint, France) was used to monitor the behavior of the mouse in the lit box. Animals were placed individually in the lit box with their heads directed toward the tunnel. Time spent in the lit box and the number of transitions between the two boxes was recorded over a 5-minute period after the first entry of the animal in the dark box. The total distance traveled in the lit box was also recorded.

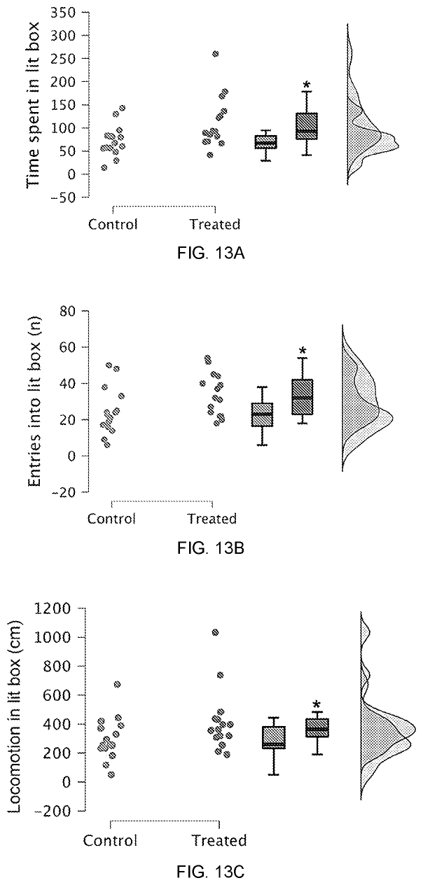

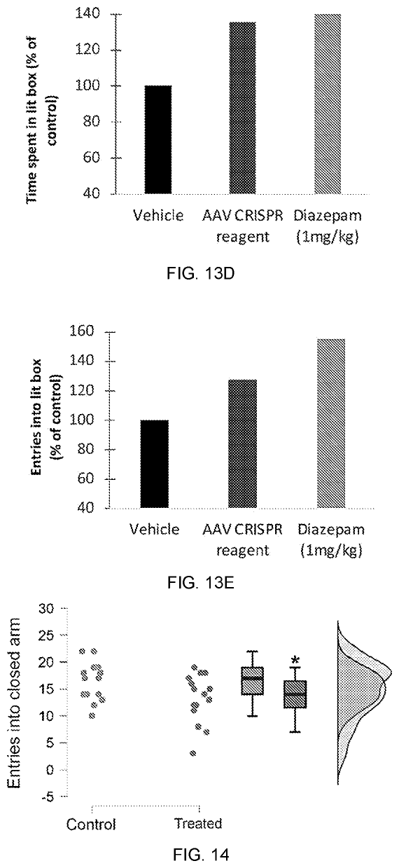

AAV-treated mice spent increased time in the lit box compared to vehicle-controls (35.7% increase, p=0.024), greater numbers of entries into the lit box (27.5% increase, p=0.024), and greater total locomotion within the lit box (27.8%, p=0.049) (FIG. 13A-13C). A regression analysis between total 5HT-2A receptor protein fluorescence and time spent in the lit box indicated a moderate negative linear correlation (r=−0.55). For context, these results were compared with the acute effects of diazepam (1 mg/kg po, 1 hour). AAV-treatment led to comparative results to diazepam in the time spent in the lit box (35.7% vs 40%) (FIG. 13D), with diazepam outperforming AAV-treated mice in the number of entries into the lit box (27.5% vs 55%) (FIG. 13E). Thus, intranasal adeno-associated virus delivery of co-packaged Cas9 DNA and a HTR2A-targeting gRNA significantly decreased anxiety and performed on par with the benzodiazepine diazepam.

### Example 10. Elevated Plus Maze Test

The ability of the designed CRISPR/Cas9 cargo to decrease anxiety utilizing an EPM test was also assessed. The EPM test is commonly used to evaluate anxiety in rodents. Male C57Bl/6J mice from Jackson Labs were utilized. Mice were received at 6-7 weeks old and allowed to acclimate for 1-2 weeks prior to testing at an average age of 8 weeks. All animals were examined, handled, and weighed prior to initiation of the study to assure adequate health and suitability. During the course of the study, 12/12 light/dark cycles was maintained. Chow and water were provided ad libitum for the duration of the study. Each mouse was randomly assigned across the treatment groups. The test was performed during the animal's light cycle phase.

The EPM has two opposite open arms and two closed arms. Mice will sometimes explore and spend time in the open arms, but anxious mice tend to first enter and spend most of the test time in the closed arms. The maze consists of two closed arms (14.5 h×5 w×35 cm length) and two open arms (6 w×35 1 cm) forming a cross, with a square center platform (6×6 cm). All visible surfaces were black acrylic. Each arm of the maze was placed on a support column 56 cm above the floor. Antistatic black vinyl curtains (7′ tall) surround the EPM to make a 5′×5″ enclosure. Animals were brought into the experimental room at least 1 hour before the test to acclimate. Mice were placed in the center of the EPM facing the closed arm for a 5-min run. All animals were tested once. The number of entries in the closed and open arms were automatically recorded by a computer, and the EPM was thoroughly cleaned after each mouse. Testing was conducted at week 5 of intranasal dosing as described above, with each mouse (n=15) receiving a total of 40 μl of AAVs equivalent to ~200 billion viral particles. Vehicle-treated animals (n=15) received 40 μL of saline for each treatment following the same protocol.

At 5 weeks post-dosing, mice exhibited a significant 24% decrease in the number of entries into the closed arm (FIG. 14) (p=0.017). In addition, the AAV-treated cohort resulted in less distance traveled into closed arms (p=0.032). There was no significant difference between the two groups in the number of entries into the open arm (p=0.50). A documented 17% increase in the time spent in open arms was observed, however this did not reach statistical significance due to the large variance observed in the AAV-treated group (p=0.257). There were no significant differences in weight between the two groups (p=0.183).

### Example 11. Vectors for Human Knockout of HTR2A

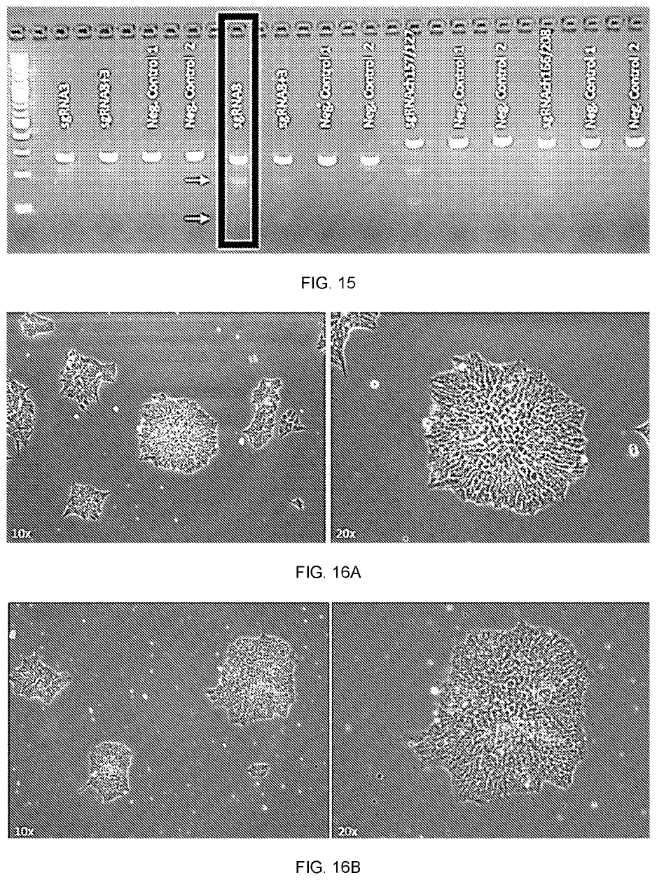

Various gRNA were designed to knockout 5HT-2A receptors in humans. gRNA targeting sequence GAUUCUGGAUGGCGACGUAG (SEQ ID NO:3) was selected for further development. This sequence has a predicted 62.37% efficiency in humans. Other test sequences displayed improved efficiency in T7E1 assays (FIG. 15), but SEQ ID NO:3 was selected for further analysis because it displays less predicted off-target activity.

Human iPSC cell line W30-1A cells (BrainXell, Wisconsin, United States) were incubated with Cas9 and the gRNA containing targeting sequence SEQ ID NO:3. Three different iPSC clones were identified, and HTR2A genomic DNA was sequenced. Each clone contained a deletion that resulted in a frameshift mutation that introduced a premature stop codon. The knockout cells were expanded on GFR MATRIGEL™ (Corning™, Arizona, United States) with regular mTeSR™ Plus media (STEMCELL™ Technologies, Canada), and the knockout cells displayed normal morphology (FIG. 16A-16B).

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

1. A method to treat symptoms of anxiety in a subject, comprising:
providing a first adeno-associated virus (AAV) vector and a second AAV vector; and
administering the first AAV vector and the second AAV vector to the subject,
wherein:
the first AAV vector is an AAV serotype 9 (AAV9) vector;
the first AAV vector comprises a first nucleic acid that comprises a first nucleotide sequence that comprises a nuclease-encoding sequence that encodes a nuclease;
the nuclease is a Cas9 nuclease that is spCas9;
the first nucleotide sequence encodes a first promoter that is operably linked to the first nucleotide sequence such that the first promoter can mediate transcription of the nuclease-encoding sequence in neurons;
the first promoter is a human methyl CpG binding protein 2 (MECP2) promoter;
the second AAV vector is an AAV9 vector;
the second AAV vector comprises a second nucleic acid that comprises a second nucleotide sequence that encodes a guide RNA (gRNA);
the second nucleotide sequence comprises a targeting sequence and a nuclease-recruiting sequence;
the targeting sequence is complementary to a genomic nucleotide sequence;
either the genomic nucleotide sequence or a reverse complement of the genomic nucleotide sequence encodes a portion of human 5-hydroxytryptamine receptor 2A (5HT-2A receptor);
either the genomic nucleotide sequence or the reverse complement is a portion of a second exon that encodes the human 5HT-2A receptor;
the targeting sequence comprises at least 90 percent sequence identity with at least 10 consecutive nucleotides set forth in GAUUCUGGAUGGCGACGUAG (SEQ ID NO. 3);
the nuclease-recruiting sequence is configured to recruit the nuclease;
the second nucleotide sequence encodes a second promoter that is operably linked to the targeting sequence and the nuclease-recruiting sequence such that the second promoter can mediate transcription of the targeting sequence and the nuclease-recruiting sequence in neurons;
the second promoter is a human U6 promoter;
the first AAV vector is administered intranasally;
at least 200 billion particles of the first AAV vector are administered to the subject;
the first AAV vector is administered in an amount sufficient to achieve a multiplicity of infection (MOI) per target cell of at least 3;
the second AAV vector is administered intranasally;
at least 200 billion particles of the second AAV vector are administered to the subject;
the second AAV vector is administered in an amount sufficient to achieve a MOI per target cell of at least 3;
the administering results in a frameshift mutation in the second exon of human 5HT-2A receptor;
the frameshift mutation results in a premature stop codon that inhibits translation of a third exon of human 5HT-2A receptor such that the translation results in a non-functional human 5HT-2A receptor;
the first AAV vector and the second AAV vector are administered in amounts sufficient to reduce expression of functional human 5HT-2A receptor in the brain of the subject by at least 20 percent; and
reduced expression of functional human 5HT-2A receptor in the brain of the subject treats the symptoms of anxiety.

2. A method to modulate brain activity in a brain of a subject, comprising:
providing a first adeno-associated virus (AAV) vector and a second AAV vector; and
administering the first AAV vector and the second AAV vector to the brain of the subject,
wherein:
the first AAV vector comprises a first nucleic acid that comprises a first nucleotide sequence that comprises a nuclease-encoding sequence that encodes a nuclease;
the second AAV vector comprises a second nucleic acid that comprises a second nucleotide sequence that encodes a guide RNA (gRNA);
the second nucleotide sequence comprises a targeting sequence and a nuclease-recruiting sequence;
the targeting sequence is complementary to a genomic nucleotide sequence that encodes a portion of a protein;
the nuclease-recruiting sequence is configured to recruit the nuclease; and
the administering results in a mutation that affects translation of the protein in the brain, which modulates brain activity in the subject.

3. The method as claimed in claim 2, wherein:
the first AAV vector is an AAV serotype 9 (AAV9) vector; and
the second AAV vector is an AAV9 vector.

4. The method as claimed in claim 2, wherein:
the nuclease is a Cas9 nuclease; and
the Cas9 nuclease is spCas9.

5. The method as claimed in claim 2, wherein the protein is a G protein-coupled receptor.

6. The method as claimed in claim 5, wherein:
binding of the G protein-coupled receptor to a neurotransmitter activates phospholipase C to cause calcium release; and
the administering modulates brain activity in the subject by modulating neurotransmitter-dependent calcium release in neurons that contain the mutation.

7. The method as claimed in claim 2, wherein the protein is 5-hydroxytryptamine receptor 2A (5HT-2A receptor).

8. The method as claimed in claim 5, wherein:
binding of the G protein-coupled receptor to a neurotransmitter inhibits adenylyl cyclase to decrease cAMP; and
the administering modulates brain activity in the subject by modulating neurotransmitter-dependent cAMP concentration in neurons that contain the mutation.

9. The method as claimed in claim 2, wherein the protein is 5-hydroxytryptamine receptor 1A (5HT-1A receptor).

10. The method as claimed in claim 5, wherein:
the mutation introduces a premature stop codon into a gene that encodes the protein;
the premature stop codon inhibits the expression of at least one exon of the gene; and
the at least one exon encodes a C-terminal intracellular region of the G protein-coupled receptor.

11. The method as claimed in claim 2, wherein:
the targeting sequence is GAUUCUGGAUGGCGACGUAG (SEQ ID NO. 3); and
the gRNA has at least 60 percent efficiency at causing the mutation in human neurons.

12. The method as claimed in claim 2, wherein the targeting sequence comprises at least 90 percent sequence identity with at least 10 consecutive nucleotides set forth in GAUUCUGGAUGGCGACGUAG (SEQ ID NO. 3).

13. The method as claimed in claim 2, comprising:
administering the first AAV vector in an amount sufficient to achieve a multiplicity of infection (MOI) per target cell of at least 3; and
administering the second AAV vector in an amount sufficient to achieve a MOI per target cell of at least 3,
wherein:
the first AAV vector is administered intranasally; and
the second AAV vector is administered intranasally.

14. The method as claimed in claim 1, comprising administering an olfactory test to the subject prior to administering the first AAV vector and the second AAV vector, wherein one or both of the first AAV vector and the second AAV vector are administered intranasally.

15. The method as claimed in claim 1, comprising focusing pulsed ultrasound in regions of the brain, wherein the regions of the brain in which the pulsed ultrasound is focused display an increased prevalence of mutation per neuron relative to other regions of the brain in which the pulsed ultrasound is not focused.

16. The method as claimed in claim 1, comprising focusing repetitive Transcranial Magnetic Stimulation in regions of the brain, wherein the regions of the brain in which the repetitive Transcranial Magnetic Stimulation is focused display an increased prevalence of mutation per neuron relative to other regions of the brain in which the repetitive Transcranial Magnetic Stimulation is not focused.

17. The method as claimed in claim 15, wherein the region comprises at least a portion of one or more of the amygdala, dorsal amygdala, hippocampus, hypothalamus, interpeduncular nucleus, anterior cingulate cortex, dorsal anterior cingulate cortex, posterior cingulate cortex, prefrontal cortex, ventromedial prefrontal cortex, pons, pontine nuclei, cerebellum, Purkinje layer of the cerebellum, orbital gyrus, and caudate nucleus.

18. A method to treat a mental health condition in a subject, comprising:
determining that the subject presents with the mental health condition; and
performing the method as claimed in claim 1,
wherein:
the mental health condition is stress, anxiety, attention deficit disorder (ADD), obsessive-compulsive disorder (OCD), post-traumatic stress disorder (PTSD), bipolar disorder, a personality disorder, memory concern, dementia, or inattention.

19. A recombinant nucleic acid, comprising a targeting sequence that comprises at least 90 percent sequence identity with at least 10 consecutive nucleotides set forth in GAUUCUGGAUGGCGACGUAG (SEQ ID NO. 3) or a reverse complement thereof.

20. The recombinant nucleic acid of claim 19, comprising a nuclease-recruiting sequence.

21. An adeno-associated virus comprising the nucleic acid of claim 19.


SEQUENCE LISTING (3 sequences)

SEQ ID NO: 1 - DNA, 23 bp, synthetic construct
  source 1..23: mol_type = other DNA
tgcaattaggtgacgactcgagg

SEQ ID NO: 2 - DNA, 200 bp, synthetic construct
  source 1..200: mol_type = other DNA
gaagacaatatctccctgagctcaattccaaactccttaatgcaattaggtgactagtgaattcaacttgtttattgcagcttataatggttacaaataaagcaatagcatcacaaatttcacaaataaagcatttttttcactgctcgaggctctaccctaatgacttcaactccagggatgctaacacttccgaagcc

SEQ ID NO: 3 - RNA, 20 nt, synthetic construct
  source 1..20: mol_type = other RNA
gattctggatggcgacgtag